(19) United States
(12) Patent Application Publication
(10) Pub. No.: US 2023/0225817 A1
(43) Pub. Date: Jul. 20, 2023

(54) DEVICES AND METHODS FOR STRESS/STRAIN ISOLATION ON A FORCE SENSOR UNIT

(71) Applicant: Intuitive Surgical Operations, Inc., Sunnyvale, CA (US)

(72) Inventors: Zhou YE, Santa Clara, CA (US); Lawrence KERVER, Lighthouse Point, FL (US); Harsukhdeep RATIA, Los Altos Hills, CA (US); Ashwinram SURESH, San Jose, CA (US)

(73) Assignee: Intuitive Surgical Operations, Inc., Sunnyvale, CA (US)

(21) Appl. No.: 17/925,209

(22) PCT Filed: May 17, 2021
(86) PCT No.: PCT/US2021/032739

§ 371 Date: Nov. 14, 2022

Related U.S. Application Data
(60) Provisional application No. 63/026,321, filed on May 18, 2020.

(51) Int. Cl. A61B 34/00; G01L 5/1627

(52) U.S. Cl. CPC A61B 34/76; G01L 5/1627; A61B 2017/00477

(57) ABSTRACT

A medical device includes a shaft comprising a proximal end portion and a distal end portion. A beam has a proximal end portion, a distal end portion, and a middle portion, and one or more strain sensors are on the middle portion. The proximal end portion of the beam is matingly coupled to the distal end portion of the shaft to form an interface. The beam comprises a discontinuity between the interface and the middle portion of the beam. In some embodiments, the medical device includes a link and the distal end portion of the beam is matingly coupled to the link to form a second interface. A second discontinuity is between the second interface and the middle portion of the beam. In some embodiments, an anchor is coupled to the shaft and the proximal end portion of the beam is matingly coupled to the anchor.

### CROSS-REFERENCE TO RELATED APPLICATIONS

This application claims priority to and the benefit of U.S. Provisional Patent Application No. 63/026,321, entitled “Devices and Methods for Stress/Strain Isolation on a Force Sensor Unit,” filed May 18, 2020, the disclosure of which is incorporated herein by reference in its entirety.

### BACKGROUND

The embodiments described herein relate to medical devices, and more specifically to endoscopic tools. More particularly, the embodiments described herein relate to devices that include strain sensors on a cantilever beam coupled to an end effector used to measure forces applied to the end effector during a medical procedure.

Known techniques for Minimally Invasive Surgery (MIS) employ instruments to manipulate tissue that can be either manually controlled or controlled via computer-assisted teleoperation. Many known MIS instruments include a therapeutic or diagnostic end effector (e.g., forceps, a cutting tool, or a cauterizing tool) mounted on a wrist mechanism at the distal end of a shaft. During an MIS procedure, the end effector, wrist mechanism, and the distal end of the shaft are inserted into a small incision or a natural orifice of a patient to position the end effector at a work site within the patient's body. The optional wrist mechanism can be used to change the end effector's orientation with respect to the shaft to perform the desired procedure at the work site. In known instruments, motion of the instrument as a whole provides mechanical degrees of freedom (DOFs) for movement of the end effector and the wrist mechanisms generally provide the desired degrees of freedom (DOFs) for movement of the end effector in relation to the shaft of the instrument. For example, for forceps or other grasping tools, known wrist mechanisms are often able to change the pitch and yaw of the end effector with reference to the shaft. A wrist may optionally provide a roll DOF for the end effector, or the roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

To enable the desired movement of the wrist mechanism and end effector, known instruments include cables that extend through the shaft of the instrument and that connect the wrist mechanism to a mechanical structure that can be used to move the cables to operate the wrist mechanism. For robotic systems, the mechanical structure is typically motor driven and operably coupled to a processing system to provide a user interface for a clinical user (e.g., a surgeon) to control the instrument.

Patients benefit from continual efforts to improve the effectiveness of MIS methods and tools. For example, reducing the size and/or the operating footprint of the shaft and wrist mechanism can allow for smaller entry incisions and reduced need for space at the surgical site, thereby reducing the negative effects of surgery, such as pain, scarring, and undesirable healing time. But, producing small medical instruments that implement the clinically desired functions for minimally invasive procedures is challenging. Specifically, simply reducing the size of known wrist mechanisms by scaling down the components will not result in an effective solution because required component and material properties do not scale. For example, efficient implementation of a wrist mechanism is complicated because the cables must be carefully routed through the wrist mechanism to maintain cable tension throughout the range of motion of the wrist mechanism and to minimize the interactions (or coupling effects) of one rotation axis upon another axis. Further, pulleys and/or contoured surfaces are generally needed to reduce cable friction, and such reduced cable friction extends instrument life and permits operation without excessive forces being applied to the cables or other structures in the wrist mechanism. Increased localized forces that may result from smaller structures (including the cables and other components of the wrist mechanism) can result in undesirable lengthening (e.g., “stretch” or “creep”) of the cables during storage and use, reduced cable life, and the like.

Some known systems include force sensing and associated haptic feedback during a MIS procedure, which brings better immersion, realism, and intuitiveness to a surgeon performing the procedure. For effective haptics rendering and accuracy, force sensors are placed on a surgical instrument and as close to the anatomical tissue interaction as possible. One approach is to include a force sensor with electrical strain sensors (e.g., strain gauges) at a distal end of a surgical instrument shaft to measure strain imparted to the surgical instrument.

One example of a force sensor unit includes a cantilever beam attached between the instrument distal tip component (e.g., in some cases a clevis or other wrist or end effector component) and the instrument shaft that extends back to the mechanical structure. Strain sensors are on the beam and are used to sense strain in X- and Y-directions (arbitrary Cartesian directions that are orthogonal to each other and to a longitudinal axis of the beam and instrument shaft). For example, the strain sensors can include full-Wheatstone bridges (full-bridges). A strain sensor bridge circuit is a circuit topology of electrical circuit in which two circuit branches (usually in parallel with each other) are bridged by a third branch connected between the first two branches at some intermediate point along them. Two full-bridges are formed on each of two adjacent orthogonal side faces of the beam to measure forces orthogonal to the longitudinal axis of the beam in both the X- and Y-directions. In some cases, the strain sensors are split into two sets, one on the distal end of the beam and the other on the proximal end of the beam in order to reject common-modes. Because the beam is secured to a distal portion of the instrument shaft, the strain sensors sense strain on the surfaces of the beam that are parallel to a longitudinal axis of the shaft. Those strains can be induced, for example, by forces that are applied orthogonal to a longitudinal axis of the shaft. A force applied orthogonal to a side face of the beam (i.e., an X or Y force) is determined by subtracting strain measurements determined by the full-bridges at the proximal and distal end portions of that side face of the beam.

A strain sensor may experience a variety of different strain sources including: the orthogonal force of interest to be measured, moment, off axis force, off axis moment, compression/tension, torsion, ambient temperature and gradient temperature. Each of the full-bridges cancel the following strains: temperature, torsion, off axis force, and off axis moment. Thus, each individual full-bridge output indicates strain due to force, moment, and compression/tension. The subtraction of an output value produced by a proximal full-bridge formed on a side face from an output value produced by a distal full-bridge on the same side face, cancels the moment and compression/tension, resulting in an output value that represents the orthogonal force of interest to be measured. The separation between the two sets of strain sensors (i.e., the strain sensors at the distal end of the beam and the strain sensors at the proximal end of the beam) determines the sensitivity of the force sensor unit, and the larger the separation the better the sensitivity. The overall length of the beam, as well as its cross-sectional area, are constrained by the space available inside the instrument shaft and the stiffness requirement of the device. Thus, efforts to reduce the overall instrument size can result in decreased separation between the strain sensors.

Additionally, regions of stress concentration, strain overload, and nonlinear distribution of stress along the length of the beam occur at the interfaces between the beam and the components to which the beam is coupled (i.e., the instrument shaft and the clevis or end effector). As such, the strain sensors need to be placed outside of these regions so as to perform force-sensing properly and avoid strain sensor damage from strain overload. Therefore, it is desirable for the design of the force sensor unit to meet two competing requirements—(i) positioning the strain sensors far enough away from the interfaces at the ends of the beam, and (ii) maximizing the separation between the two sets of strain sensors.

The force sensor unit also needs to perform force sensing in a consistent manner as the instrument shaft rolls over a full 360 degrees. That means the output signals from the X- and Y-gauges should follow the same sinusoidal distribution over the full range of roll motion. This need for consistent force sensing poses some specific requirements on the cross-sectional shape of the cantilever beam.

Thus, a need exists for improved endoscopic tools that have force-sensing capabilities and that can address the above-mentioned problems associated with stress concentration, strain overload, and nonlinear distribution of forces that occur at the interfaces between the beam and components to which the beam is joined.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter. In some embodiments, a medical device includes a shaft and a beam. The shaft comprises a proximal end portion and a distal end portion. The beam has a proximal end portion, a distal end portion, and a middle portion between the proximal end portion of the beam and the distal end portion of the beam. One or more strain sensors are on the middle portion of the beam. The proximal end portion of the beam is matingly coupled to the distal end portion of the shaft to form an interface. The beam comprises a discontinuity between the interface and the middle portion of the beam.

In some embodiments, the medical device further includes an anchor coupled to the distal end portion of the shaft. The anchor comprises a coupling portion and the proximal end portion of the beam is matingly coupled to the coupling portion of the anchor to couple the distal end portion of the beam to the distal end portion of the shaft.

In some embodiments, the discontinuity is a first discontinuity, the interface is a first interface and the medical device further includes a link. The distal end portion of the beam is matingly coupled to the link to form a second interface, and the beam comprises a second discontinuity between the second interface and the middle portion of the beam. In some embodiments, the first discontinuity includes the middle portion of the beam having a first cross-sectional area and the proximal end portion of the beam having a second cross-sectional area, with the first cross-sectional area being different than the second cross-sectional area. The second discontinuity includes the distal end portion having a third cross-sectional area, with the first cross-sectional area being different than the third cross-sectional area.

In some embodiments, the first discontinuity includes the proximal end portion of the beam being tapered, and the tapered proximal end portion being a guide for alignment of the beam to the anchor along the center axis of the shaft. In some embodiments, the second discontinuity includes the distal end portion of the beam being tapered, and the tapered distal end portion being a guide for alignment of the beam to the link along the center axis of the shaft. In some embodiments, the first discontinuity includes a first recessed region between the middle portion of the beam and the proximal portion of the beam, and the second discontinuity includes a second recessed region between the middle portion of the beam and the distal end portion of the beam.

In some embodiments, the proximal end portion of the beam comprises a shape to be received in only one orientation within an opening of the anchor having a first corresponding shape, and the distal end portion of the beam comprises a shape to be received in only one orientation within an opening of the link having a second corresponding shape. In some embodiments, a proximal end face of the middle portion of the beam is spaced apart from a distal end face of the anchor when the beam is coupled to the anchor, and a distal end face of the middle portion of the beam is spaced apart from a proximal end face of the link when the beam is coupled to the link.

In some embodiments, a medical device comprises a shaft, a beam, an anchor and a link. The shaft comprises a proximal end portion and a distal end portion, and a center axis extending between the proximal end portion and the distal end portion. The beam has a proximal end portion, a distal end portion and a middle portion between the proximal end portion and the distal end portion. The proximal end portion of the beam has a first cross-sectional area, the middle portion of the beam has a second cross-sectional area, and the distal end portion of the beam has a third cross-sectional area. The first cross-sectional area being different than the second cross-sectional area and the third cross-sectional area. One or more strain sensors are on the middle portion of the beam. The anchor is coupled to the distal end portion of the shaft, and comprises a coupling portion. The proximal end portion of the beam is matingly coupled to the coupling portion of the anchor. The link comprises a coupling portion, and the distal end portion of the beam is matingly coupled to the coupling portion of the link.

In some embodiments, the coupling portion of the anchor is an opening configured to receive the proximal end portion of the beam, and the coupling portion of the link is an opening configured to receive the distal end portion of the beam. In some embodiments, the proximal end portion of the beam is tapered and the distal end portion of the beam is tapered. The proximal end portion is a guide for alignment of the beam to the anchor along the center axis of the shaft, and the distal end portion is a guide for alignment of the beam to the link along the center axis of the shaft.

In some embodiments, the proximal end portion of the beam comprises a shape to be received in only one orientation within an opening of the anchor having a first corresponding shape, and the distal end portion of the beam comprises a shape to be received in only one orientation within an opening of the link having a second corresponding shape. In some embodiments, the proximal end portion of the beam comprises a D-shape and is configured to be received within a corresponding D-shaped opening of the anchor. In some embodiments, the distal end portion of the beam comprises a D-shape and is configured to be received within a corresponding D-shaped opening of the link. In some embodiments, the proximal end portion of the beam is welded to the anchor and the distal end portion of the beam is welded to the link.

In some embodiments, the medical device further comprises an outer shaft, and the shaft extends within a lumen of the outer shaft. In some embodiments, the shaft is movable relative to the outer shaft. In some embodiments, the outer shaft translates longitudinally along the center axis relative to the shaft and the beam. In some embodiments, the shaft can rotate relative to outer shaft.

In some embodiments, the one or more strain sensors includes a first strain sensor on the middle portion of the beam at a first location on the middle portion of the beam, and a second strain sensor on the middle portion of the beam at a second location on the middle portion of the beam at a spaced longitudinal distance from the first strain sensor.

In some embodiments, the first cross-sectional area of the middle portion of the beam has a length and a width defining a first outer face, a second outer face, a third outer face and a fourth outer face. The first outer face and the second outer face being oriented perpendicular to the third outer face and the fourth outer face, and the first strain sensor is on the first face and the second strain sensor is on the first face.

In some embodiment, a medical device comprises a shaft, a link, a beam and one or more sensors. The shaft comprises a proximal end portion and a distal end portion. The distal end portion of the shaft defines a first mating opening and the link defines a second mating opening. The beam has a proximal end portion, a distal end portion and a middle portion between the proximal end portion and the distal end portion. The proximal end portion of the beam is tapered and is coupled within the first mating opening. The distal end portion of the beam is tapered and is coupled within the second mating opening. The one or more sensors is on the middle portion of the beam. In some embodiments, the one or more sensors is one or more strain sensors.

In some embodiments, the above medical device further comprises an outer shaft, and the shaft extends within a lumen of the outer shaft and is movable relative to the outer shaft. In some embodiments, the outer shaft translates longitudinally relative to the shaft and the beam. In some embodiments, the shaft can rotate relative to outer shaft. In some embodiments, the one or more sensors is one or more strain sensors and includes a first strain sensor on the middle portion of the beam at a first location on the middle portion of the beam, and a second strain sensor on the middle portion of the beam at a second location on the middle portion of the beam at a spaced longitudinal distance from the first strain sensor.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of an optional auxiliary unit of the minimally invasive teleoperated surgery system shown in FIG. 1.

FIG. 3 is a perspective view of a user control console of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5 is a diagrammatic illustration of a portion of a medical device including a force sensor unit, according to an embodiment.

FIGS. 6A-6D are each schematic illustrations of a different embodiment of a force sensor unit.

FIG. 7 is a perspective view of a medical device according to an embodiment.

FIG. 8A is an enlarged perspective view of a distal end portion of the medical device of FIG. 7.

FIG. 8B is an enlarged perspective view of a distal end portion of the medical device of FIG. 7 with the outer shaft removed.

FIG. 9A is a perspective view of a distal portion of the medical device of FIG. 7.

FIG. 9B is a perspective distal end view of the shaft and anchor of the medical device of FIG. 7.

FIG. 10 is a side view of the beam, distal link and anchor of the medical device of FIG. 7.

FIGS. 11 and 12 are each an exploded view of the beam, distal link and anchor of FIG. 10 showing a distal perspective (FIG. 11) and a proximal perspective (FIG. 12).

FIGS. 13A and 13B are a side view (FIG. 13A) and a perspective view (FIG. 13B) of the beam of the medical device of FIG. 7.

FIG. 14 is a perspective view of the beam of the medical device of FIG. 7 illustrating strain sensors mounted thereto.

FIG. 15 is an enlarged perspective view of a proximal end portion of the beam of FIG. 11.

FIG. 16A is a graph illustrating stress versus a length of a beam as generated by a computer model for a medical device, according to an embodiment.

FIGS. 16B and 16C are enlarged views of a left-side portion (FIG. 16B) and a right-side portion (FIG. 16C) of the graph of FIG. 16A.

FIG. 17A is a side view of a portion of a medical device according to another embodiment.

FIG. 17B is a side view of a portion of the medical device of FIG. 17A with the outer shaft removed for illustrative purposes.

FIG. 18A is side view of a beam of the medical device of FIG. 17A.

FIG. 18B is a cross-sectional view of the beam of FIG. 18A.

FIGS. 19 and 20 are each a different perspective view of a distal portion of the medical device of FIG. 17B with the outer sheath removed.

FIGS. 21A and 21B are each a different exploded perspective view of the beam and a proximal clevis of the medical device of FIG. 17A.

FIG. 22 is a perspective view of the proximal end of the beam of the medical device of FIG. 17A.

FIG. 23 is a perspective view of the beam of the medical device of FIG. 17A.

### DETAILED DESCRIPTION

The embodiments described herein can advantageously be used in a wide variety of grasping, cutting, and manipulating operations associated with minimally invasive surgery. The medical instruments or devices of the present application enable motion in three degrees of freedom (e.g., about a pitch axis, a yaw axis, and a grip axis). The embodiments described herein further can be used to determine the forces exerted on (or by) a distal end portion of the instrument during use.

The medical instruments described herein. can include a force sensor unit having a cantilevered beam and one or more strain sensors on the beam. The beam can have a discontinuity between an active portion of the beam where the strain sensors are placed and at least one of the interfaces between the beam and the connected mating components. Further, the interface(s) can be moved outside of the active portion of the beam, while maintaining the overall length of the cantilever. The discontinuity between the active portion of the beam (with the sensors) and the interface portions of the beam (where the beam connects to mating components) can isolate the beam from undesired stresses/strains caused by mechanical interactions with other mating parts. As a result, the stress forces exerted on the beam maintain a substantially linear distribution along the length of the beam, and any stress concentrations or strain overloads are moved to a portion of the beam outside of the active portion of the beam.

As described herein, such a discontinuity can be formed by changing the cross-sectional area of the beam along its length, and in particular, providing the active portion of the beam with a cross-sectional area than portions of the beam outside of the active portion. For example, in some embodiments, the beam can be provided with at least one end having a cross-sectional area that is different than a cross-sectional area of the active portion of the beam. That end can couple the beam to a mating component of the medical instrument, such as a distal end component (e.g., a link, an end effector, etc.). The interface portion of the beam (e.g., at the distal end portion) now becomes the contact interface between the beam and the other mating components, so that the stress raisers and possible strain overload will be moved towards the interface portions of the beam rather than on the active portion of the beam.

A discontinuity can be formed in a variety of different manners. For example, in some embodiments, a discontinuity is formed by the interface end(s) of the beam being tapered, and the tapered end can be coupled to a mating component such as a distal end component or link. In such embodiments, the tapered ends can also serve to help align the mating components during assembly. For example, in some embodiments, the tapered ends can be received in a corresponding opening in the mating component to which the beam is to be coupled. The tapered shape of the ends can help guide insertion of the ends of the beam into the openings. Thus, the tapered ends can ensure that the center axis of the beam is aligned with the center axis of the instrument shaft, thereby improving the accuracy of the force measurements.

In some embodiments, the discontinuity can be formed by providing a recess region between the active portion of the beam and the interface portion of the beam. For example, an interface portion of the beam can include a section that has a smaller cross-sectional area than the active portion of the beam and also include an interface portion (e.g., a distal end or proximal end portion) that couples to the mating components (e.g., instrument shaft on proximal end or distal component on distal end). That coupling portion can have the same or different cross-sectional area as the active portion of the beam.

In some embodiments, a discontinuity is provided at both a proximal end and a distal end of the beam. For example, the beam can include a proximal interface portion that couples to, for example, an inner shaft of the surgical instrument (or another component of the surgical instrument), and a distal interface portion that couples to a distal end component of the surgical instrument. In some embodiments, both the proximal end interface portion and the distal end interface portion have a cross-sectional area that is different than a cross-sectional area of the active portion of the beam. In some embodiments, both the proximal end interface portion and the distal end interface portion are tapered. In some embodiments, one or both interface end portions have the same shape of the active portion of the beam (e.g., round, square, rectangular, triangular, etc.) but have a different cross-sectional area than the active portion of the beam. In some embodiments, the proximal end portion of the beam can have the same shape and/or cross-sectional area as the distal end portion of the beam. In some embodiments, the proximal end portion of the beam can have a different shape and/or a different cross-sectional area as the distal end portion of the beam. For example, in some embodiments, the distal end portion of the beam can be tapered, and the proximal end portion may not be tapered but have a portion that has a different cross-sectional area than the middle portion of the beam.

In some embodiments, the active portion of the beam is formed integrally or monolithically with the interface portion(s) (e.g., a proximal interface end portion and/or a distal interface end portion). In some embodiments, the interface portion(s) can be formed as a separate component and coupled to the active portion of the beam. For example, the interface portion(s) can be welded to the active portion of the beam.

In some embodiments, to achieve uniform signal outputs over a full range of instrument roll, the cross-sectional shape of the beam is identical per every 90-degree rotation about its center axis. In an embodiment having tapered interfaces the interfaces can serve to not only provide for stress isolation, but also for improving the concentricity (axial alignment) between the beam and its mating parts as described in more detail below.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

Aspects of the invention are described primarily in terms of an implementation using a da Vinci® Surgical System, commercialized by Intuitive Surgical, Inc. of Sunnyvale, Calif. Examples of such surgical systems are the da Vinci Xi® Surgical System (Model IS4000), da Vinci X® Surgical System (Model IS4200), and the da Vinci Si® Surgical System (Model IS3000). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® Surgical Systems (e.g., the Model IS4000, the Model IS3000, the Model IS2000, the Model IS1200) are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

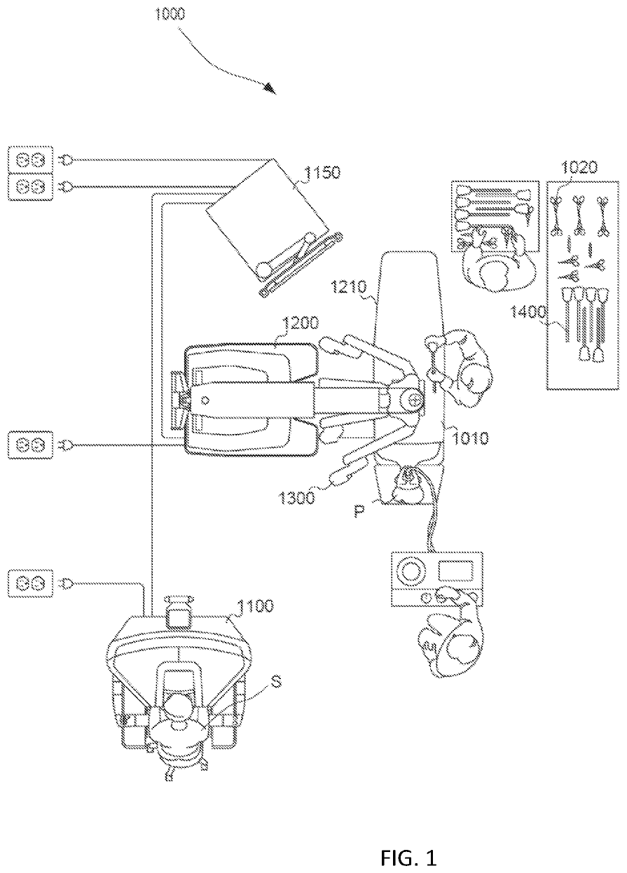

FIG. 1 is a plan view illustration of a computer-assisted teleoperation system. Shown is a medical device, which is a Minimally Invasive Robotic Surgical (MIRS) system 1000 (also referred to herein as a minimally invasive teleoperated surgery system), used for performing a minimally invasive diagnostic or surgical procedure on a Patient P who is lying on an Operating table 1010. The system can have any number of components, such as a user control unit 1100 for use by a surgeon or other skilled clinician S during the procedure. The MIRS system 1000 can further include a manipulator unit 1200 (popularly referred to as a surgical robot), and an optional auxiliary equipment unit 1150. The manipulator unit 1200 can include an arm assembly 1300 and a tool assembly removably coupled to the arm assembly. The manipulator unit 1200 can manipulate at least one removably coupled instruments 1400 (also referred to herein as a “tool”) through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the MIRS 1000.

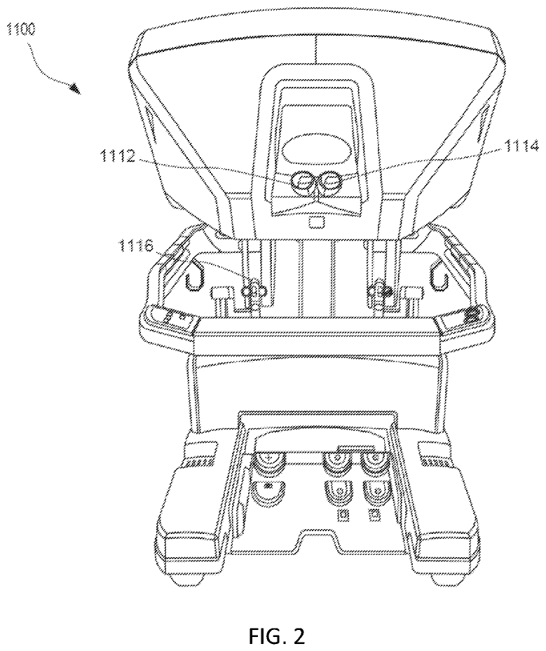

FIG. 2 is a perspective view of the control unit 1100. The user control unit 1100 includes a left eye display 1112 and a right eye display 1114 for presenting the surgeon S with a coordinated stereo view of the surgical site that enables depth perception. The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, strain and/or tactile feedback sensors (not shown) may be employed to transmit position, force, and tactile sensations from the instruments 1400 back to the surgeon's hands through the input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments however, the user control unit 1100 and the surgeon S can be in a different room, a completely different building, or other remote location from the patient allowing for remote surgical procedures.

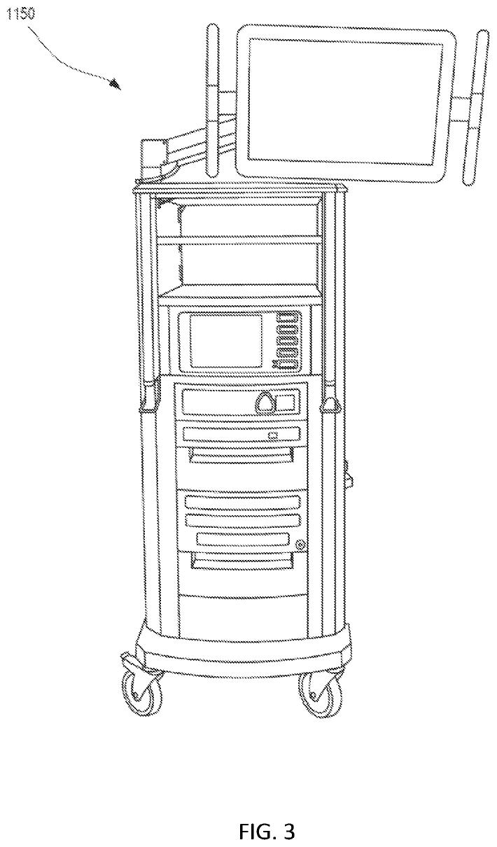

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

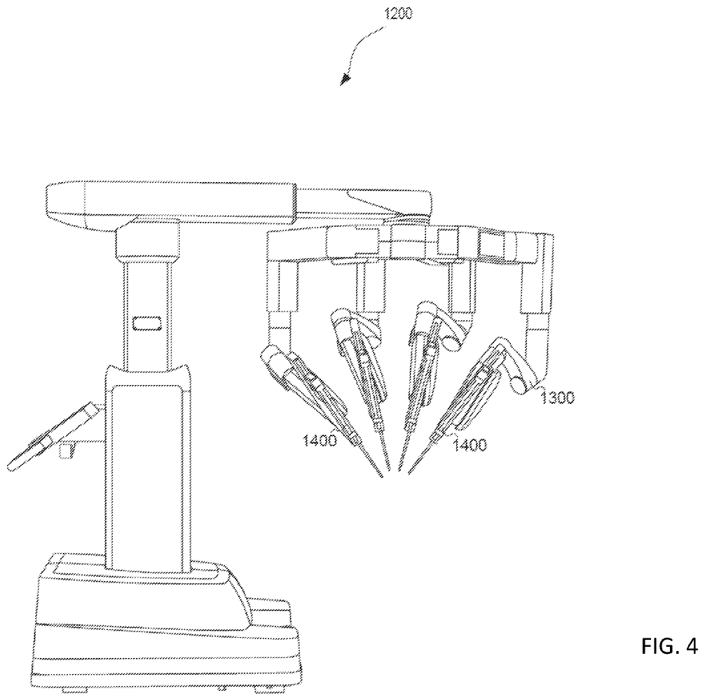

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having a number of joints. Moreover, the instruments 1400 and the imaging device are positioned and manipulated through incisions or natural orifices in the patient P in a manner such that a software and/or kinematic remote center of motion is maintained at the incision or orifice. In this manner, the incision size can be minimized.

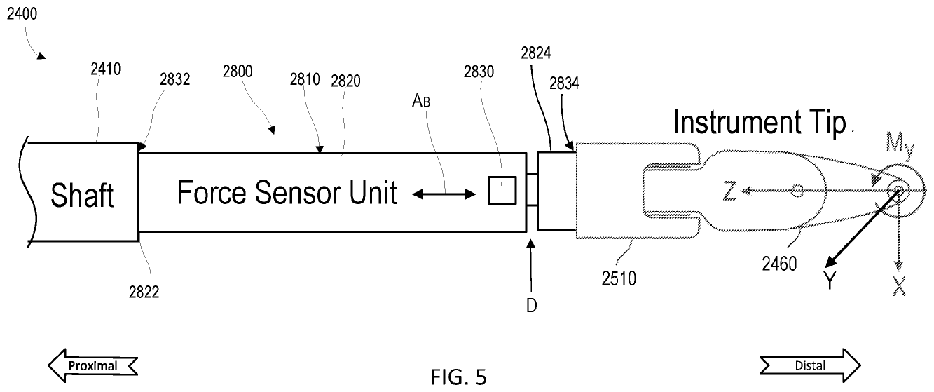

FIG. 5 is a schematic illustration of a portion of a distal end portion of a medical instrument 2400, according to an embodiment. The surgical instrument 2400 includes a shaft 2410, a force sensor unit 2800 including a beam 2810, with one or more strain sensors (e.g., strain gauges) 2830 mounted on a surface along the beam 2810, and an end effector 2460 coupled at a distal end portion of the surgical instrument 2400. The end effector 2460 can include, for example, articulatable jaws or another suitable surgical tool that is coupled to a link 2510. In some embodiments, the link 2510 can be included within a wrist assembly having multiple articulating links. The shaft 2410 includes a distal end portion that is coupled to a proximal end portion 2822 of the beam 2810 to form a first interface 2832. In some embodiments, the distal end portion of the shaft 2410 is coupled to the proximal end portion 2822 of the beam via another coupling component (such as an anchor or coupler, not shown). The shaft 2410 can also be coupled at a proximal end portion to a mechanical structure (not shown in FIG. 5) configured to move one or more components of the surgical instrument, such as, for example, the end effector 2460. The mechanical structure can be similar to the mechanical structure 7700 described in more detail below with reference to medical instrument 7400.

The beam 2810 includes a middle portion 2820 (which functions as an active portion of the beam), a proximal end portion 2822 and a distal end portion 2824. The beam 2800 defines a beam center axis A_{B}, which can be aligned within a center axis of the instrument shaft 2410. As shown, the strain sensor 2830 is coupled to the middle portion 2820 of the beam 2810. Thus, the middle portion 2820 functions as the active portion of the beam 2810 to sense the forces applied to a distal end portion of the instrument 2400. Although shown as including only one strain sensor 2830, in other embodiments, the beam 2810 can include any number of strain sensors in any of the arrangements as described herein. The distal end portion 2824 of the beam 2810 is coupled to the end effector 2460 via a link 2510. Specifically, the distal end portion 2824 is matingly coupled to the link 2510 to form a second interface 2834. In some embodiments, the link 2510 can be, for example, a clevis of the end effector 2460. In this embodiment, a discontinuity D is formed at the second interface 2834 between the distal end portion 2824 of the beam 2800 and the link 2510. In other embodiments, a discontinuity can be formed at the first interface 2832. In yet other embodiments a discontinuity can be formed at both the first interface 2832 and the second interface 2834. The discontinuity D can isolate the middle portion 2820 of the beam 2810 from undesired stresses/strains caused by mechanical interactions resulting from the mating coupling to the link 2510, as described in more detail below.

Generally, during a medical procedure, the end effector 2460 contacts anatomical tissue, which may result in X, Y, or Z direction forces being imparted on the end effector 2460 and that may result in moment forces such as a moment M_{Y} about a y-direction axis as shown in FIG. 5. The one or more strain sensors 2830, which can be strain gauges, can be used to produce signals induced by forces in the X, Y and Z directions. The signals can be used to measure strain in the beam 2810 which can be used to determine forces imparted on the end effector 2460 in the X and Y axes directions. For example, the signals of a distal set of the strain sensors 2830 can be subtracted from the signals of a proximal set of strain sensors 2830. Any signals induced by the Z-direction forces would be identical on the two sets of strain sensors and would be eliminated after the subtraction. The X and Y axes forces are transverse (e.g., perpendicular) to the Z axis (which is parallel or collinear with the center axis A_{B}). Such transverse forces acting upon the end effector 2460 can cause a slight bending of the beam 2810 (about either or both of the X axis or the Y axis), which can result in a tensile strain imparted to one side of the beam 2810 and a compression strain imparted to the opposite side of the beam 2810. The strain sensors 2830 on the beam 2810 can measure such tensile and compression strains.

In some embodiments, the instrument 2400 (or any of the instruments described herein) can include additional force sensors to measure the axial force(s) (i.e., in the direction of the Z-axis parallel to the beam center axis A_{B}) imparted on the end effector 2460. An axial force sensor in an example surgical instrument can comprise a deflectable planar diaphragm sensor that deflects in response to a force. Alternatively, a ferrite core within an inductive coil or a fiber Bragg grating formed within an optical fiber can be used, for example. Other axial force sensor designs may be used to sense a resilient axial displacement of the shaft 2410 (e.g., relative to the proximally mounted mechanical structure, not shown). An axial force F_{Z} imparted to the end effector 2460 can cause axial displacement of the shaft 2410 in a direction along a center axis of the shaft (substantially parallel to the beam center axis A_{B}). The axial force F_{Z} may be in the proximal direction (e.g., a reactive force resulting from pushing against tissue with the end effector) or it may be in the distal direction (e.g., a reactive force resulting from pulling tissue grasped with the end effector).

As described above, the X and Y forces imparted on the end effector 2460 can result in stress concentrations and strain overloads at the interfaces between the beam 2810 and its mating components, which in this embodiment includes the shaft 2410 and the link 2510. Similarly stated, imperfections and non-uniformity of the coupling between the distal end 2824 of the beam 2810 and the link 2510, which result from part-to-part variability, normal manufacturing tolerances, or the like, can produce nonlinear stresses. Such stresses, in turn, can lead to erroneous strain readings from the strain sensors. As described above, the discontinuity D is provided to shift any such stress concentrations and strain overloads outside of the middle portion 2820 (i.e., active portion) of the beam 2810 to the distal end portion 2824 at the second interface 2834 between the distal end portion 2824 and the link 2510. Said another way, the discontinuity D functions to isolate the middle portion 2820 from the undesired stresses/strains caused by the coupling at the second interface 2834. In this embodiment, the discontinuity D is provided by a recessed region formed between the middle portion 2820 and the distal end portion 2824 of the beam 2800. In other words, the distal end portion 2824 includes a first section having a first cross-sectional area that is smaller than the cross-sectional area of the middle portion 2820 of the beam 2800 and an interface portion that couples to the link 2510. The interface portion can include a cross-sectional area that is the same or different than the middle portion 2820 of the beam 2810. Thus, a discontinuity of the cross-sectional area along the length of the beam 2810 has been formed between the middle portion 2820 and the distal end portion 2824 of the beam 2810.

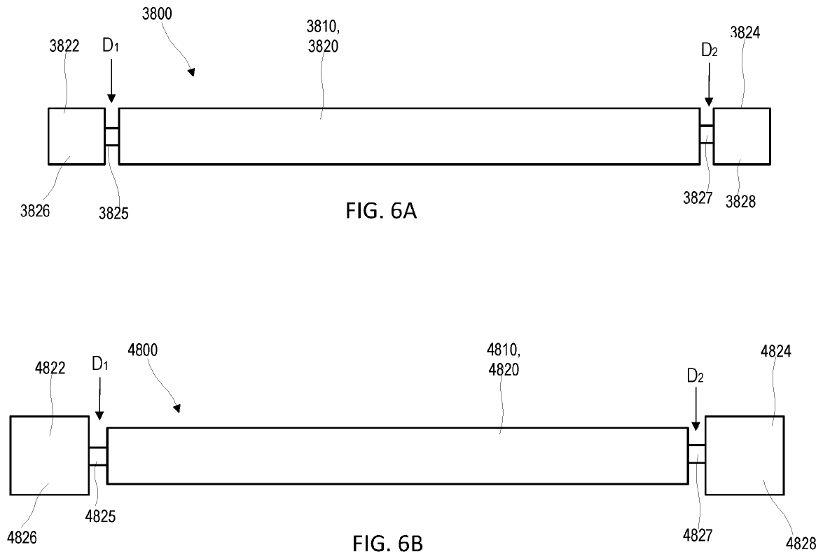

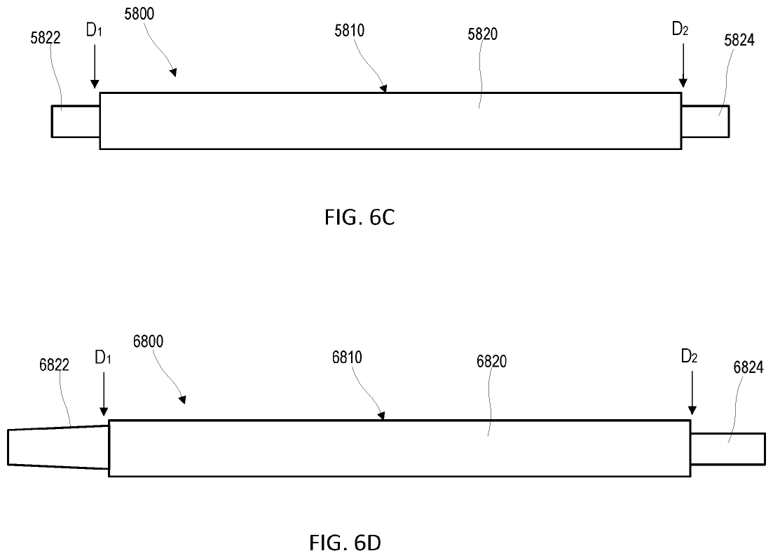

FIGS. 6A-6C are schematic illustrations of alternative embodiments of a force sensor unit that can be included in any of the surgical instruments described herein. FIG. 6A illustrates a force sensor unit 3800 that includes a beam 3810 having a middle portion 3820 (which functions as the active portion of the beam), a proximal end portion 3822 and a distal end portion 3824. Although not shown, one or more strain sensors can be coupled to the middle portion 3820. The proximal end portion 3822 can be coupled to, for example a shaft of a surgical instrument as described herein (e.g., the shaft 2410 or the shaft 7410). The distal end portion 3824 can be coupled to, for example, a distal component of the surgical instrument such as, for example, an end effector, a link of an end effector, a distal tool, etc. For example, the distal end portion 3824 can be coupled to the link 2510 or the link 7510. In this embodiment, a first discontinuity D₁ is formed between the proximal end portion 3822 of the beam 3810 and the middle portion 3820 of the beam 3810 and a second discontinuity D₂ is formed between the distal end portion 3824 and the middle portion 3820 of the beam 3810.

In this embodiment, the discontinuity D₁ and the discontinuity D₂ are each provided in the form of a recessed region formed between the proximal end portion 3822 and the middle portion 3820 and between the distal end portion 3824 and the middle portion 3820, respectively. As described above, to form the recessed regions, the proximal end portion 3822 and the distal end portion 3824 can each include a first section, 3825 and 3827, respectively, having a first cross-sectional area that is smaller than the cross-sectional area of the middle portion 3820 of the beam 3810 and smaller than the cross-sectional area of a second portion (which functions as an interface portion), 3826 and 3828, respectively, that couples to a mating component. In this embodiment, the second portions 3826 and 3828 of the proximal end portion 3822 and the distal end portion 3824, respectively, each have a cross-sectional area that is substantially the same as the middle portion 3820 of the beam 3810.

In other embodiments, the cross-sectional area (or size) of the second portions can be different than the cross-sectional area (or size) of the middle portion. For example, FIG. 6B illustrates a force sensor unit 4800 that includes a beam 4810 having a middle portion 4820 (which functions as the active portion of the beam), a proximal end portion 4822 and a distal end portion 4824. Although not shown, one or more strain sensors can be coupled to the middle portion 4820. The proximal end portion 4822 can be coupled to, for example a shaft of as surgical instrument as described herein (e.g., the shaft 2410 or the shaft 7410). The distal end portion 4824 can be coupled to, for example, a distal component of the surgical instrument such as, for example, an end effector, a link of an end effector, a distal tool, etc. (e.g., the link 2510 or the link 7510). In this embodiment, a first discontinuity D₁ is formed between the proximal end portion 4822 of the beam 4810 and the middle portion 4820 of the beam 4810 and a second discontinuity D₂ is formed between the distal end portion 4824 and the middle portion 4820 of the beam 4810.

In this embodiment, the discontinuity D₁ and the discontinuity D₂ are each provided in the form of a recessed region formed between the proximal end portion 4822 and the middle portion 4820 and between the distal end portion 4824 and the middle portion 4820, respectively. As described above, to form the recessed regions, the proximal end portion 4822 and the distal end portion 4824 can each include a first section, 4825 and 4827, respectively, having a first cross-sectional area that is smaller than the cross-sectional area of the middle portion 4820 of the beam 4810 and smaller than the cross-sectional area of a second portion (which functions as an interface portion), 4826 and 4828, respectively, that couples to a mating component. In this embodiment, the second portions 4826 and 4828 of the proximal end portion 4822 and the distal end portion 4824, respectively, each have a cross-sectional area that is different than the middle portion 4820 of the beam 4810.

FIG. 6C illustrates a force sensor unit 5800 that includes a beam 5810 having a middle portion 5820 (which functions as the active portion of the beam), a proximal end portion 5822 and a distal end portion 5824. Although not shown, one or more strain sensors can be coupled to the middle portion 5820. The proximal end portion 5822 can be coupled to, for example a shaft of as surgical instrument as described herein (e.g., the shaft 2410 or the shaft 7410). The distal end portion 5824 can be coupled to, for example, a distal component of the surgical instrument such as, for example, an end effector, a link of an end effector, a distal tool, etc. (e.g., the link 2510 or the link 7510). In this embodiment, a first discontinuity D₁ is formed between the proximal end portion 5822 of the beam 5810 and the middle portion 5820 of the beam 5810 and a second discontinuity D₂ is formed between the distal end portion 5824 and the middle portion 5820 of the beam 5810.

In this embodiment, the discontinuity D₁ is provided by the proximal end portion 5822 having a different cross-sectional area (i.e., smaller) than a cross-sectional area of the middle portion 5820. The discontinuity D₂ is provided by the distal end portion 5824 having a different cross-sectional area (i.e., smaller) than the cross-sectional area of the middle portion 5820.

FIG. 6D illustrates a force sensor unit 6800 that includes a beam 6810 having a middle portion 6820 (which functions as the active portion of the beam), a proximal end portion 6822 and a distal end portion 6824. Although not shown, one or more strain sensors can be coupled to the middle portion 6820. The proximal end portion 6822 can be coupled to, for example a shaft of as surgical instrument as described herein (e.g., the shaft 2410 or the shaft 7410). The distal end portion 6824 can be coupled to, for example, a distal component of the surgical instrument such as, for example, an end effector, a link of an end effector, a distal tool, etc. (e.g., the link 2510 or the link 7510). In this embodiment, a first discontinuity D₁ is formed between the proximal end portion 6822 of the beam 6810 and the middle portion 6820 of the beam 6800 and a second discontinuity D₂ is formed between the distal end portion 6824 and the middle portion 6820 of the beam 6810.

In this embodiment, the discontinuity D₁ is provided by the proximal end portion 6822 having a different cross-sectional area (i.e., smaller) than a cross-sectional area of the middle portion 6820. The discontinuity D₂ is provided by the distal end portion 6824 having a different cross-sectional area (i.e., smaller) than the cross-sectional area of the middle portion 6820. In addition, in this embodiment, the distal end portion 6824 has a constant cross-sectional area along its length and the proximal end portion 6822 is tapered (i.e., has a cross-section that is monotonically decreasing at a constant rate along its length). The tapered shape of the proximal end portion 6822 can facilitate alignment of the beam 6810 to its mating component (e.g., an instrument shaft). For example, the tapered proximal end portion 6822 can be matingly coupled within a portion of the shaft and can facilitate alignment of the center axis of the beam 6810 and the center axis of the shaft.

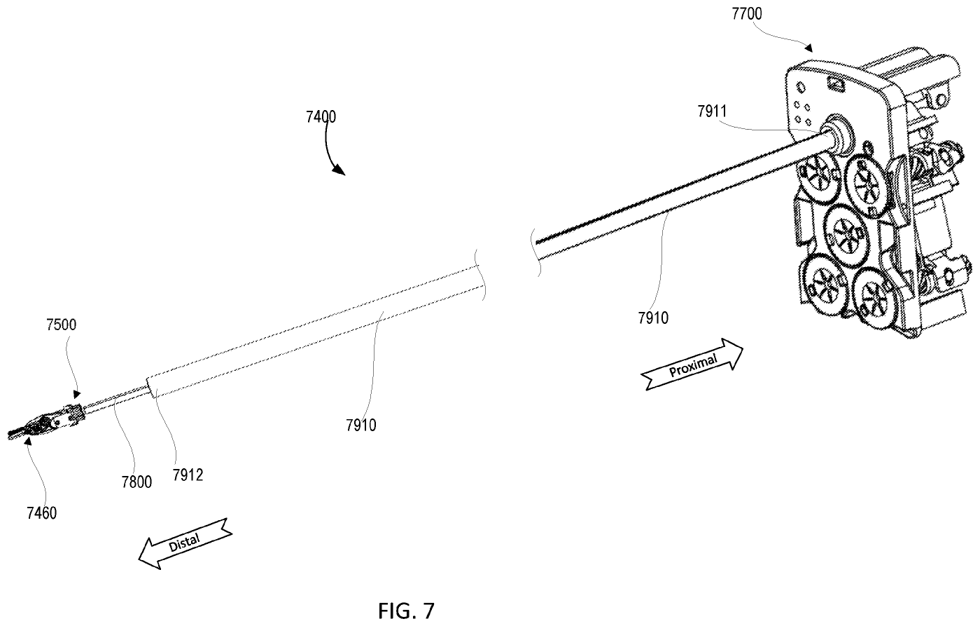

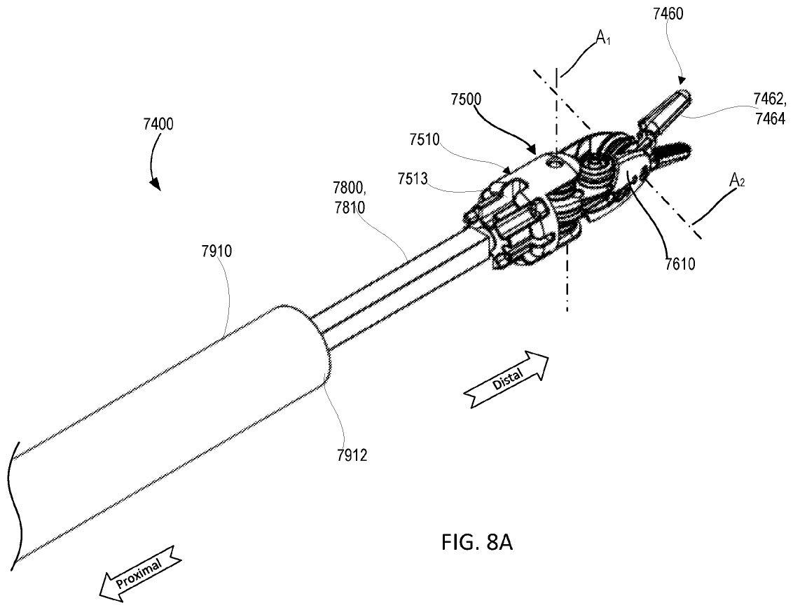

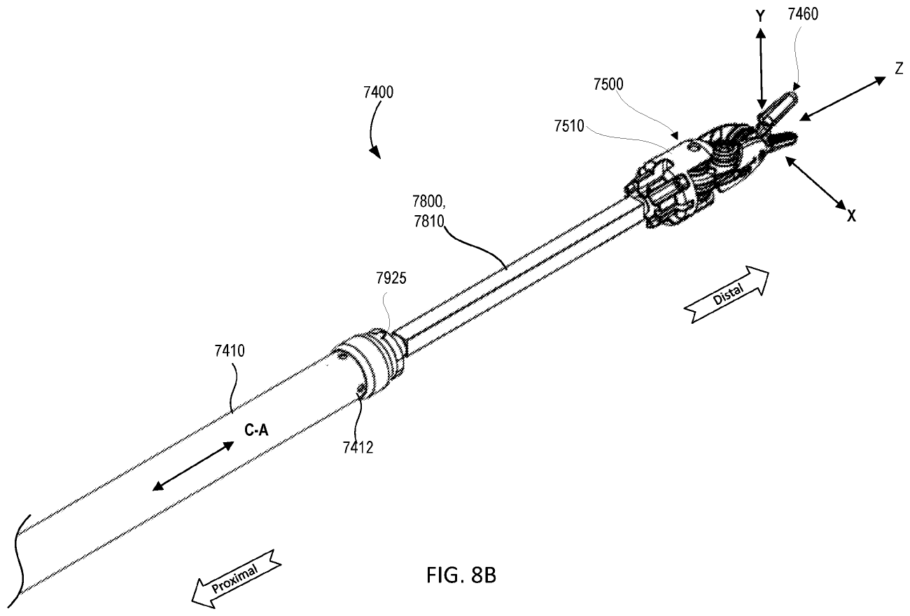

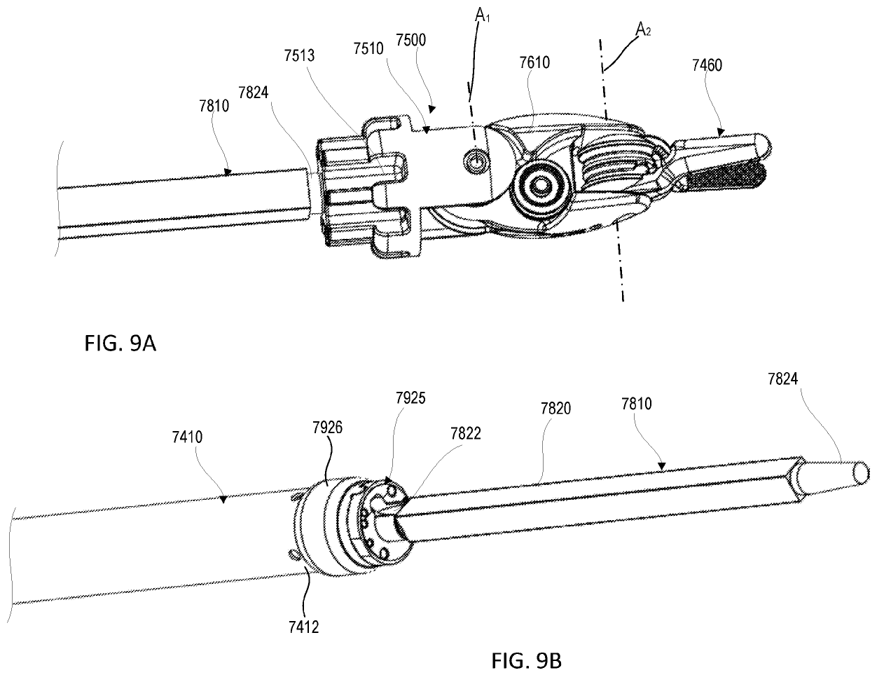

FIGS. 7-15 are various views of a medical instrument 7400, according to an embodiment. In some embodiments, the instrument 7400 or any of the components therein are optionally parts of a surgical system that performs surgical procedures, and which can include a manipulator unit, a series of kinematic linkages, a series of cannulas, or the like. The instrument 7400 (and any of the instruments described herein) can be used in any suitable surgical system, such as the MIRS system 1000 shown and described above. The instrument 7400 includes a mechanical structure 7700, an outer shaft 7910, a shaft 7410 (see FIGS. 8B and 9, which functions as an inner shaft in this embodiment), a force sensor unit 7800 that includes a beam 7810, a wrist assembly 7500, and an end effector 7460. Although not shown, the instrument 7400 can also include a number of cables that couple the mechanical structure 7700 to the wrist assembly 7500 and end effector 7460. The instrument 7400 is configured such that select movements of the cables produces rotation of the wrist assembly 7500 (i.e., pitch rotation) about a first axis of rotation A₁ (see FIG. 8A) (which functions as a pitch axis, the term pitch is arbitrary), yaw rotation of the end effector 7460 about a second axis of rotation A₂ (see FIG. 8A) (which functions as the yaw axis, the term yaw is arbitrary), a cutting rotation of the tool members of the end effector 7460 about the second axis of rotation A₂, or any combination of these movements. Changing the pitch or yaw of the instrument 7400 can be performed by manipulating the cables in a similar manner as described, for example, in U.S. Pat. No. 8,821,480 B2 (filed Jul. 16, 2008), entitled “Four-Cable Wrist with Solid Surface Cable Channels,” and U.S. Pat. No. 6,394,998 B1 (filed Sep. 17, 1999), entitled “Surgical Tools for use in Minimally Invasive Telesurgical Applications,” each of which is incorporated herein by reference in its entirety. Thus, the specific movement of each of the cables to accomplish the desired motion is not described below.

The shaft 7410 includes a proximal end (not shown) that is coupled to the mechanical structure 7700, and a distal end 7412 (see FIGS. 8B and 9) that is coupled to the beam 7810 via an anchor 7925. In some embodiments, the proximal end of the shaft 7410 is coupled to the mechanical structure 7700 in a manner that allows movement of the shaft 7410 along a center axis C-A of the shaft 7410 (shown in FIG. 8B) relative to the mechanical structure 7700. Allowing the shaft 7410 to “float” in the Z direction facilitates measurement of forces along the Z axis, as described herein. In some embodiments, the proximal end of the shaft 7410 can be movably coupled to the mechanical structure 7700 via a four bar linkage of the types shown and described in International Patent Appl. No. PCT/US2019/061883 (filed Nov. 15, 2019), entitled “Surgical Instrument with Sensor Aligned Cable Guide,” which is incorporated herein by reference in its entirety. A bushing 7914 is disposed at the distal end 7412 of the shaft 7410 adjacent the anchor 7925. The bushing 7914 is on the outer portion of the inner shaft 7410 and is therefore disposed between the shaft 7410 and the outer shaft 7910. The shaft 7410 also defines a lumen (not shown) and/or multiple passageways through which the cables and other components (e.g., electrical wires, ground wires, or the like) can be routed from the mechanical structure 7700 to the wrist assembly 7500. The anchor 7925 can be received at least partially within the lumen of the shaft 7410 and can be fixedly coupled to the shaft 7410 via an adhesive bond, a weld, or any other permanent coupling mechanism (i.e., a coupling mechanism that is not intended to be removed during normal use).

The outer shaft 7910 can be any suitable elongated shaft that can be disposed over the shaft 7410 and includes a proximal end 7911 that can be coupled to the mechanical structure 7700 and a distal end 7912. The outer shaft 7910 defines a lumen between the proximal end 7911 and the distal end 7912. The shaft 7410 extends within the lumen of the outer shaft 7910 and can move relative to the outer shaft 7910. For example, the shaft 7410 can rotate relative to the shaft 7910 and/or can translate longitudinally in a direction parallel to the center axis C-A of the shaft 7410. For example, in some embodiments, the proximal end 7911 of the outer shaft 7910 is fixedly coupled to the mechanical structure 7700 and the shaft 7410 is coupled to move relative to the mechanical structure 7700. In other embodiments, the outer shaft 7910 or portions thereof can move relative to the mechanical structure 7700 (e.g., the outer shaft 7910 can be a telescoping shaft). Thus, in some embodiments, the outer shaft 7910 is operatively coupled to the mechanical structure 7700 and can be moved or translated longitudinally relative to the shaft 7410 in a direction parallel to the center axis C-A.

The mechanical structure 7700 produces movement of the cables (not shown) to produce the desired movement (pitch, yaw, or grip) at the wrist assembly 7500. Specifically, the mechanical structure 7700 includes components and controls to move some of the cables in a proximal direction (i.e., to pull in certain cables) while simultaneously allowing the distal movement (i.e., releasing or “paying out”) of other of the cables in equal lengths. In this manner, the mechanical structure 7700 can maintain the desired tension within the cables, and in some embodiments, can ensure that the lengths of the cables are conserved (i.e., moved in equal amounts) during the entire range of motion of the wrist assembly 7500. In other embodiments, however, conservation of the lengths of the cables is not required.

In some embodiments, the mechanical structure 7700 can include one or more mechanisms that produce translation (linear motion) of a portion of the cables. Such a mechanisms can include, for example, a gimbal, a lever, or any other suitable mechanism to directly pull (or release) an end portion of any of the cables. For example, in some embodiments, the mechanical structure 7700 can include any of the mechanical structures (referred to as backend assemblies or actuators) or components described in U.S. Patent Application Pub. No. US 20157/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disk Wrist Joint,” each of which is incorporated herein by reference in its entirety. In other embodiments, however, the mechanical structure 7700 can include a capstan or other motor-driven roller that rotates or “winds” a portion of any of the bands to produce the desired band movement. For example, in some embodiments, the mechanical structure 7700 can include any of the mechanical structures (referred to as backend assemblies or actuators) or components described in U.S. Pat. No. 9,204,923 B2 (filed Jul. 16, 2008), entitled “Medical Instrument Electronically Energized Using Drive Cables,” which is incorporated herein by reference in its entirety.

Referring to FIG. 8A, the wrist assembly 7500 includes a proximal first link 7510 and a distal second link 7610. The first link 7510 includes a distal portion that is coupled to a proximal portion of the second link 7610 at a joint such that the second link 7610 can rotate relative to the first link 7510 about a first axis of rotation A₁ (which functions as the pitch axis, the term pitch is arbitrary). The proximal first link 7510 includes a proximal portion that is coupled to the beam 7810 as described in more detail below. The proximal first link 7510 defines multiple slots 7513 (see, e.g., FIGS. 8A, 9A, 10-12) along a portion of its length through which the cables for actuating the wrist assembly 7500 and end effector 7460 extend. The slots 7513 can also be used to introduce a fluid to effectuate cleaning of the instrument 7400. For example, when the outer shaft 7910 is moved to a distal position covering the beam 7810 and abutting the proximal first link 7510, the distal end portion of the slots 7513 are exposed or accessible such that a cleaning fluid can be introduced through the distal end portion of the slots 7513. When the outer shaft 7910 is moved proximally, the slots 7513 and cables therein are also accessible for cleaning purposes.

A distal end of the distal second link 7610 is coupled to the end effector 7460 such that the end effector 7460 can rotate about a second axis of rotation A₂ (see FIG. 8A) (which functions as the yaw axis (the term yaw is arbitrary)). The end effector 7460 can include at least one tool member 7462 having a contact portion 7464 configured to engage or manipulate a target tissue during a surgical procedure. For example, in some embodiments, the contact portion 7464 can include an engagement surface that functions as a gripper, cutter, tissue manipulator, or the like. In other embodiments, the contact portion 7464 can be an energized tool member that is used for cauterization or electrosurgical procedures. The end effector 7460 is operatively coupled to the mechanical structure 7700 such that the tool member 7462 rotates relative to shaft 7410 about the first axis of rotation A₁. In this manner, the contact portion 7464 of the tool member 7462 can be actuated to engage or manipulate a target tissue during a surgical procedure. The tool member 7462 (or any of the tool members described herein) can be any suitable medical tool member. Moreover, although only one tool member 7462 is identified, as shown, the instrument 7400 can include two tool members that cooperatively perform gripping or shearing functions. In other embodiments, an end effector can include more than two tool members.

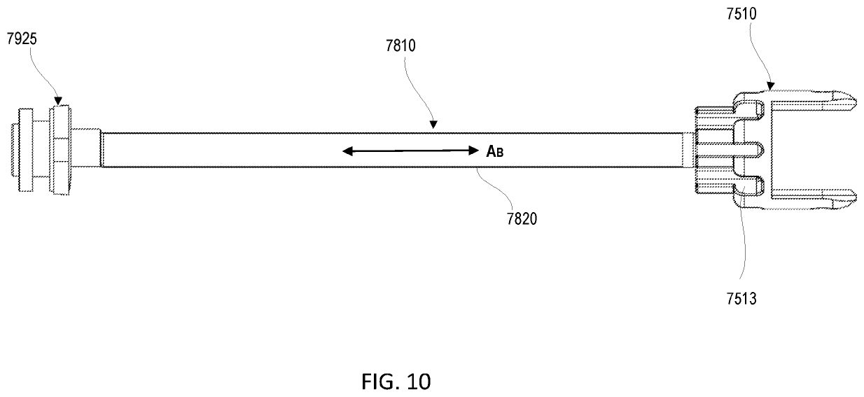

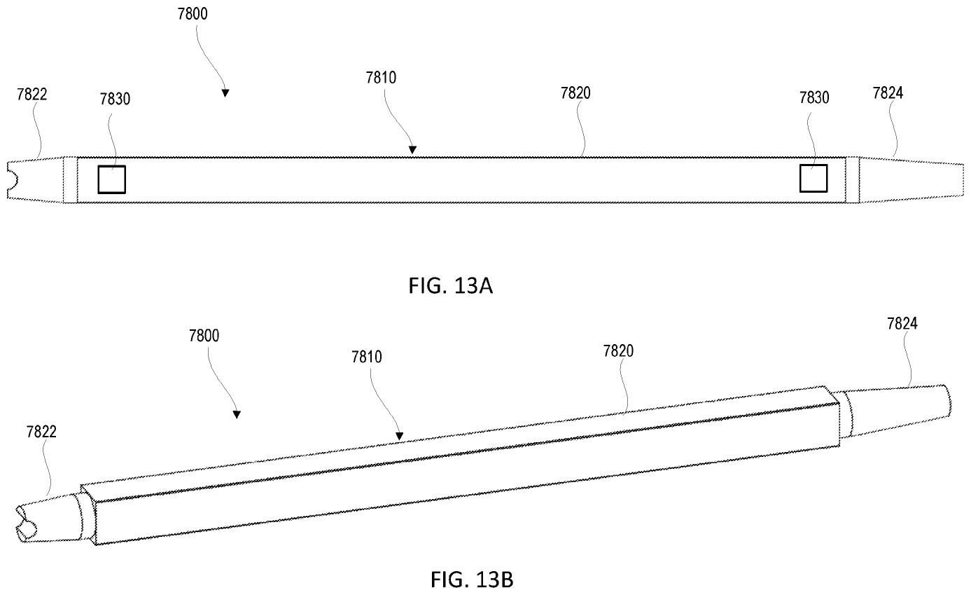

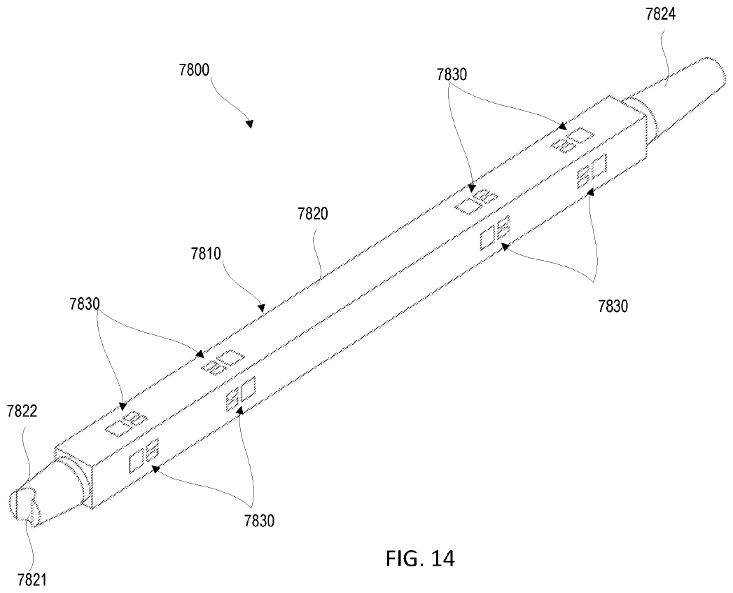

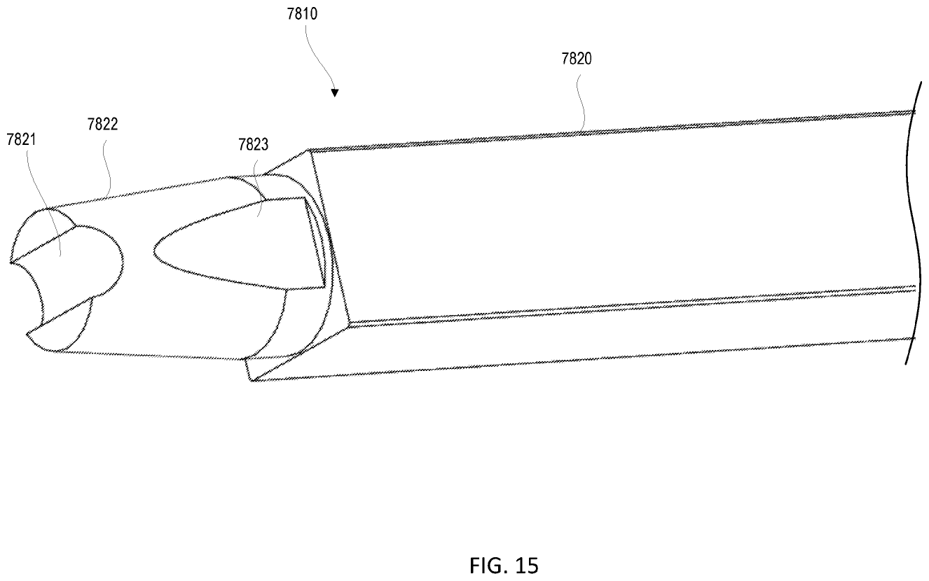

The beam 7810 includes a proximal end portion 7822, a middle portion 7820 (which functions as an active portion of the beam 7810) and a distal end portion 7824. The beam 7810 has a center axis A_{B} defined along a length of the beam 7810 (see FIG. 10). One or more strain sensors 7830 (see FIGS. 13A and 14) are mounted on the middle portion 7820 of the beam 7810. The strain sensors 7830 are not shown in some of the figures for illustrative purposes only. The strain sensors 7830 can be, for example, strain gauges, and can be used to measure forces imparted on the surgical instrument during a surgical procedure as described in more detail below. In this embodiment, the middle portion 7820 defines four side surfaces disposed perpendicular to each other and on which the strain sensors 7830 can be mounted (see FIG. 14). Such an arrangement is also described and shown in PCT Application No. PCT/US18/61113, which is incorporated herein by reference. In this embodiment, the cross-section of the middle portion 7820 is substantially square shaped. Thus, the cross-sectional shape of the middle portion 7820 is identical for every ninety degrees of rotation. In this manner, the output from the strain sensors 7830 (which are disposed on only two of the four sides of the middle portion 2820) will be consistent throughout the entire range of the roll of the shaft 7410 (i.e., rotation of the shaft 7410 about the center axis A_{B}). As shown in FIG. 14, there are multiple strain sensors 7820 on a given side surface at both the proximal end portion and the distal end portion of the middle portion 7820 of the beam 7810 for redundancy purposes. In some embodiments, there may only be a single strain sensor 7830 on a given side surface of each of the proximal end portion and the distal end portion of the middle portion 7820 of the beam 7810.

In this embodiment, both the distal end portion 7824 and the proximal end portion 7822 of the beam 7810 are tapered but each has a different cross-sectional shape and size than the other. As described above, in alternative embodiments, the proximal end portion 7822 and the distal end portion 7824 can have the same cross-sectional shape and size). In this embodiment, the proximal end portion 7822 defines an end cutout region 7821 (see FIG. 15) that is used for manufacturing purposes and also provides clearance for routing of electrical components (not shown) disposed within the shaft 7410 and anchor 7925. The proximal end portion 7822 also defines a side cutout region 7823 (see FIG. 15) that provides an entry into the lumen of the shaft 7410 (via the cutout 7927 in the anchor) to allow routing of the electrical wiring (not shown) to the strain sensors 7830. For example, the electrical wiring can extend from a proximal end of the instrument 7400 through the shaft 7410, exit the cutout 7927 and extend along the beam 7810 to the strain sensors 7830.

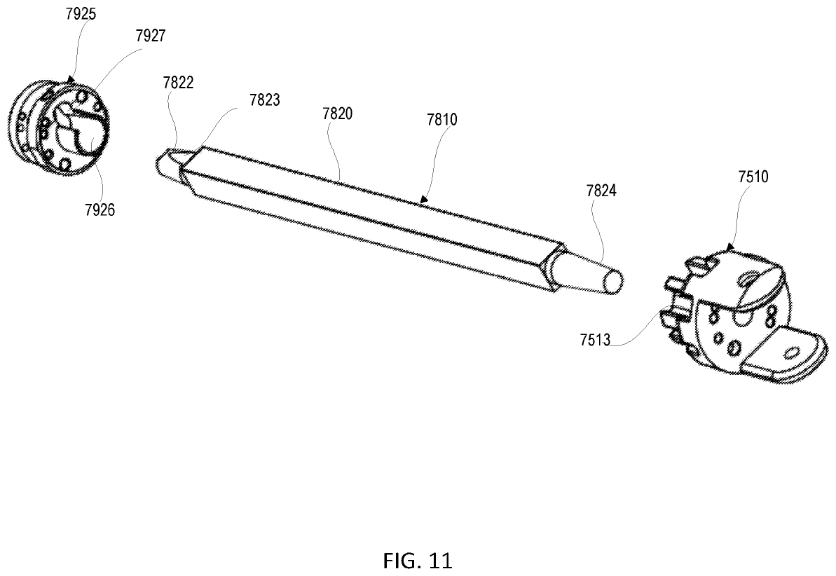

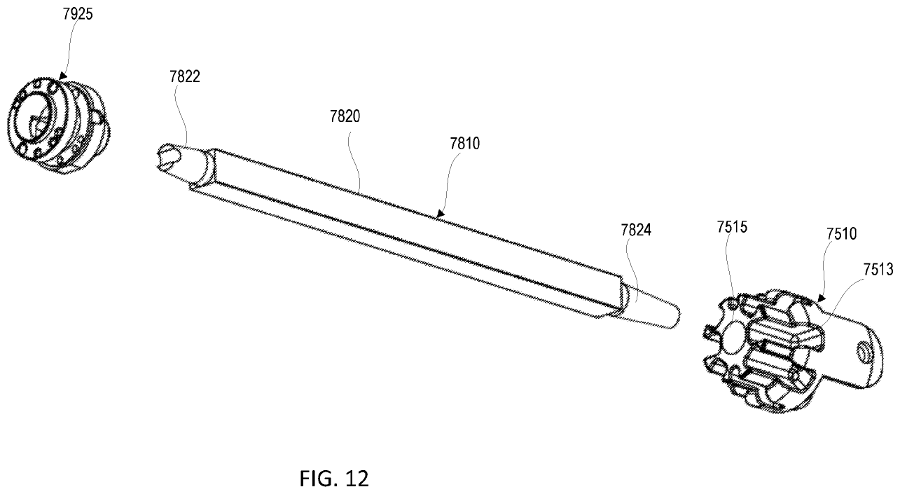

The beam 7810 is coupled to a distal end portion 7412 of the shaft 7410 via the anchor 7925 and to the proximal link 7510 of the wrist assembly 7500 (see, e.g., FIGS. 8B-10). More specifically, the anchor 7925 defines an opening 7926 (see FIG. 11) that can matingly receive the tapered proximal end portion 7822 of the beam 7810. The anchor 7925 also defines the cutout 7927 through which wires can be routed. The proximal end portion 7822 can be coupled to the anchor 7925 with, for example, welding, an adhesive or other suitable coupling methods. Similarly, the proximal link 7510 defines an opening 7515 (see FIG. 12) that can matingly receive the distal end portion 7824 of the beam 7810. The distal end portion 7824 can be coupled to the link 7510 with, for example, welding, an adhesive or other suitable coupling methods.

In use, the end effector 7460 contacts anatomical tissue, which may result in X, Y, or Z direction forces (see FIG. 8B) being imparted on the end effector 7460 and that may also result in moment forces about the various axes. As described above for instrument 2400, the strain sensors 7830 (see FIGS. 13A and 14) can be used to produce signals induced by forces in the X, Y and Z directions and the signals can be used to measure strain in the beam 7810, which can in turn be used to determine forces imparted on the end effector 7460 in the X and Y axes directions. For example, the signals of a distal set of the strain sensors 7830 can be subtracted from the signals of a proximal set of strain sensors 7830. Any signals induced by the Z-direction forces would be identical on the two sets of strain sensors and would be eliminated after the subtraction. Thus, the strain sensors 7830 can be used to determine forces imparted on the end effector 7460 that are transverse (e.g., perpendicular) to the center axis A_{B} of the beam 7810 as such forces are transferred to the beam 7810 in the X and Y directions (see FIG. 8B). Specifically, the transverse forces acting upon the end effector 7460 can cause a slight bending of the beam 7810, which can result in a tensile strain imparted to one side of the beam 7810 and a compression strain imparted to the opposite side of the beam 7810. The strain sensors 7830 are coupled to the beam 7810 to measure such tensile and compression strains.

As described above, such X and Y forces can result in stress concentrations and strain overloads at the interfaces between the beam 7810 and its mating components. Such stress concentrations and strain overloads can result in inaccurate readings at the force sensors 7830. As described herein, to increase the accuracy of the force measurements and reduce or eliminate such issues, a discontinuity is formed between the beam 7810 and the connection interfaces between the beam 7810 and its mating components. In this embodiment, the discontinuities are created by the connection of the beam 7810 to the link 7510 and the anchor 7925 by the tapered distal end portion 7824 and tapered proximal end portion 7822, respectively. More specifically, as described above, the distal end portion 7824 has a different cross-sectional area than a cross-sectional area of the middle portion 7820 of the beam 7810 on which the force sensors are mounted. This creates a first discontinuity between the distal end portion 7824 and the middle portion 7820 and provides for stress concentrations or strain overloads to be transferred to the distal interface where the distal end portion 7824 is coupled to the link 7510. Similarly, the proximal end portion 7822 has a different cross-sectional area than the cross-sectional area of the middle portion 7820 of the beam 7810, which creates a second discontinuity between the proximal end portion 7822 and the middle portion 782 and provides for stress concentrations or strain overloads to be transferred to the proximal interface where the proximal end portion 7822 is coupled to the anchor 7925.

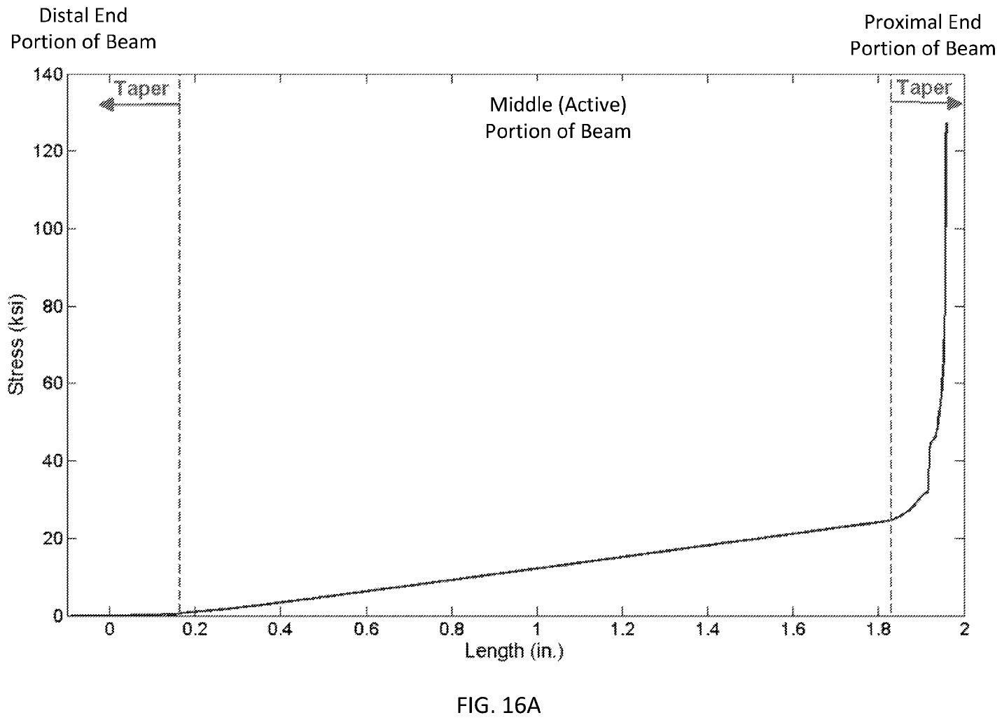

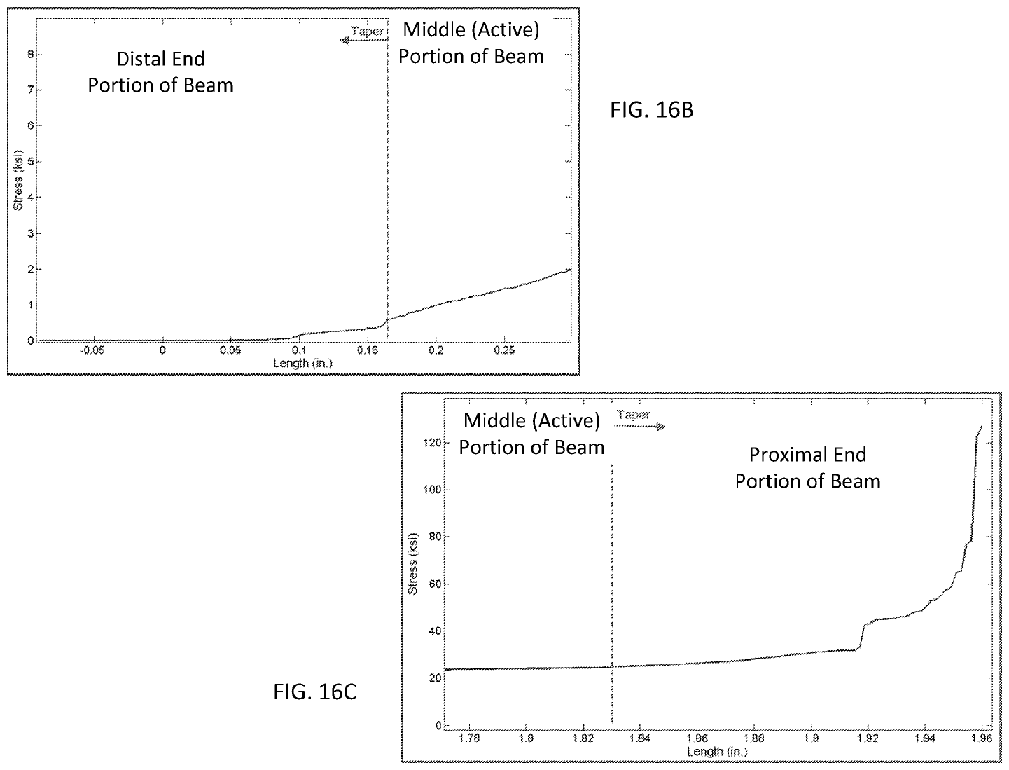

FIG. 16A is a graph showing beam stress as a function of the beam length as computer modeled for the instrument 7400. FIG. 16B is an enlarged view of the graph showing the stress at the distal end 7824 of the beam and FIG. 16C is an enlarged view of the graph showing the stress at the proximal end 7822 of the beam. As shown in FIG. 16A, it is desirable for the stress load on the beam 7810 along the middle portion (active portion) 7820 to be substantially linear along the length of the beam 7810. As best shown in FIGS. 16B and 16C, at the proximal end portion and the distal end portion of the beam, nonlinear distribution of stress forces can occur. In this example, FIG. 16B shows nonlinear behavior in the tapered distal end portion 7824 of the beam, including a nonlinear increase in the stress at the point where the taper ends. In this example, FIG. 16C illustrates nonlinear behavior and substantial stress overload at the proximal end portion 7822 of the beam. Thus, by including the first discontinuity and the second discontinuity, the nonlinear behavior and stress overload is isolated away from the active portion of the force sensing beam 7810.

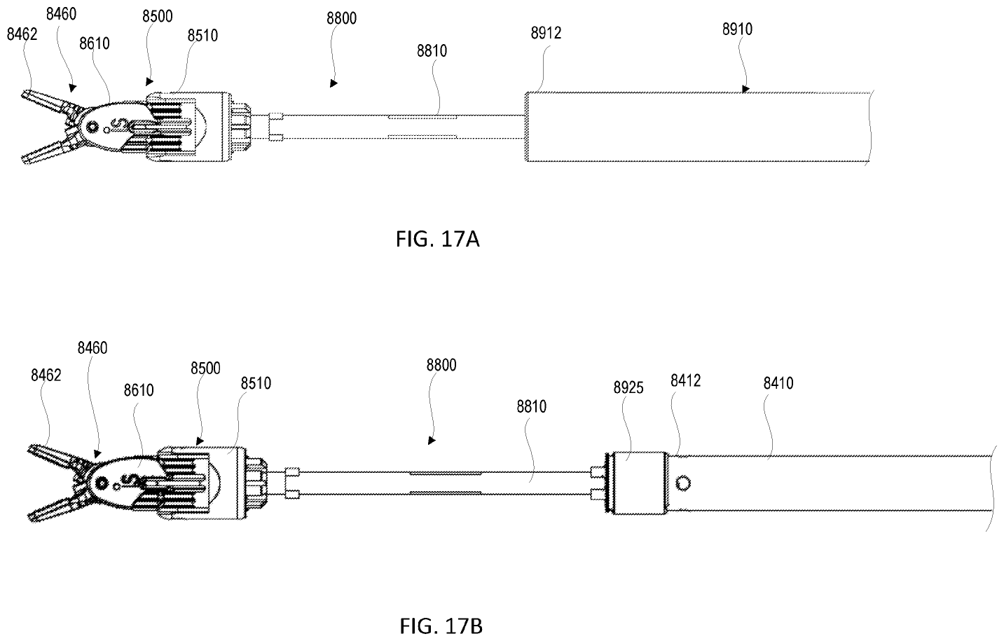

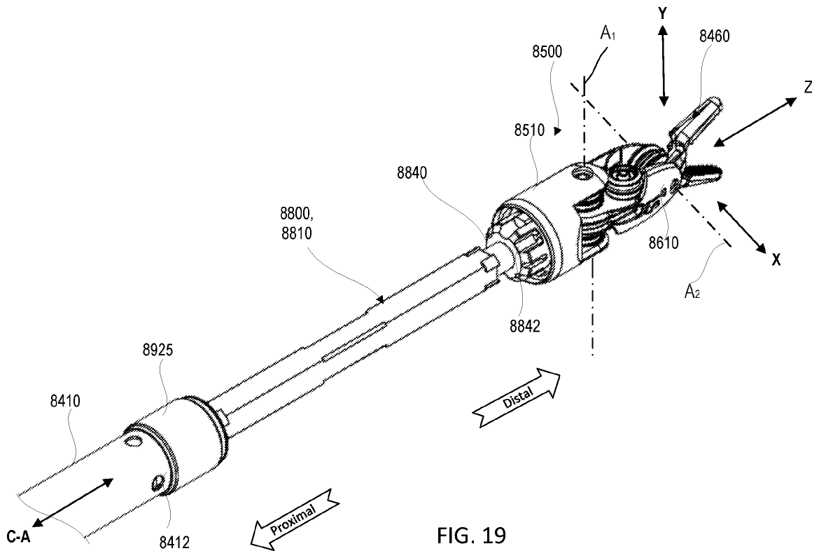

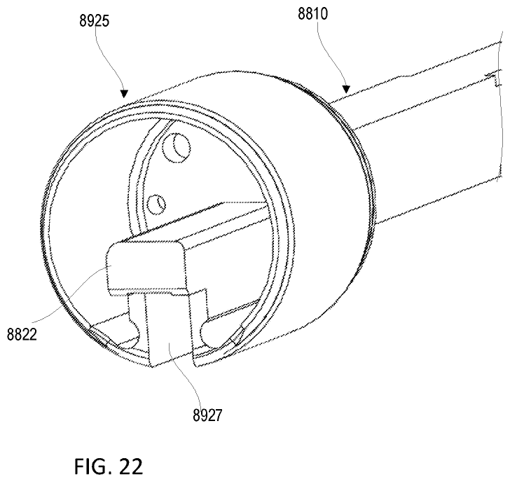

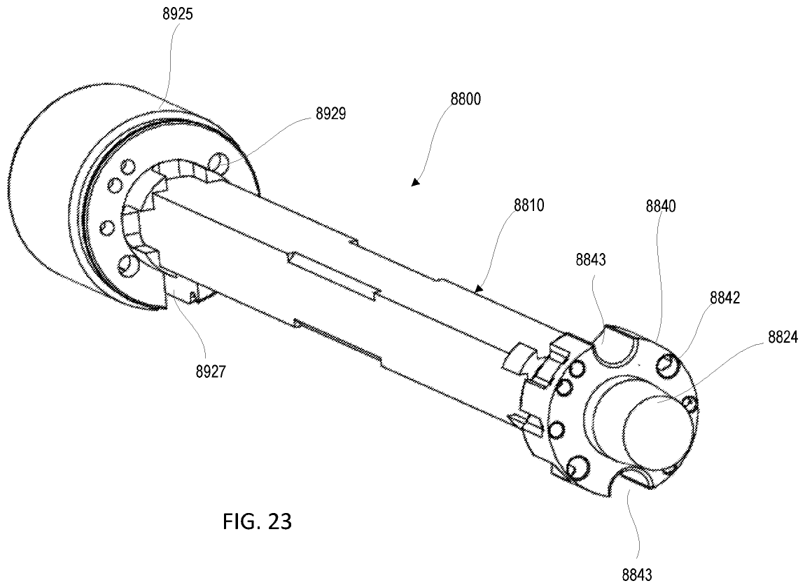

FIGS. 17A-23 are various views of a medical instrument 8400, according to an embodiment. In some embodiments, the instrument 8400 or any of the components therein are optionally parts of a surgical system that performs surgical procedures, and which can include a manipulator unit, a series of kinematic linkages, a series of cannulas, or the like. The instrument 8400 (and any of the instruments described herein) can be used in any suitable surgical system, such as the MIRS system 1000 shown and described above. The instrument 8400 includes a mechanical structure (not shown) that can be the same as or similar to the mechanical structure 7700, an outer shaft 8910, a shaft 8410, which functions as an inner shaft in this embodiment, a force sensor unit 8800 (that includes a beam 8810), a wrist assembly 8500, and an end effector 8460. Although not shown, the instrument 8400 can also include a number of cables that couple the mechanical structure to the wrist assembly 8500 and end effector 8460. The instrument 8400 is configured such that select movements of the cables produces rotation of the wrist assembly 8500 (i.e., pitch rotation) about a first axis of rotation A₁ (see FIG. 19) (which functions as a pitch axis, the term pitch is arbitrary), yaw rotation of the end effector 8460 about a second axis of rotation A₂ (see FIG. 19) (which functions as the yaw axis, the term yaw is arbitrary), a cutting rotation of the tool members of the end effector 8460 about the second axis of rotation A₂, or any combination of these movements. Changing the pitch or yaw of the instrument 8400 can be performed by manipulating the cables in a similar manner as described, for example, in U.S. Pat. No. 8,821,480 B2 (filed Jul. 16, 2008), entitled “Four-Cable Wrist with Solid Surface Cable Channels,” and U.S. Pat. No. 6,394,998 B1 (filed Sep. 17, 1999), entitled “Surgical Tools for use in Minimally Invasive Telesurgical Applications,” each of which is incorporated herein by reference in its entirety. Thus, the specific movement of each of the cables to accomplish the desired motion is not described below.

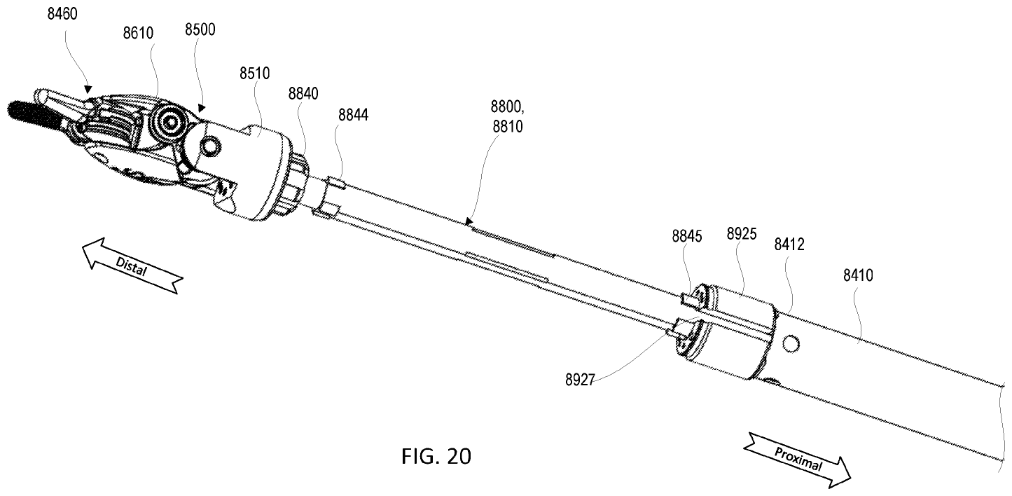

The shaft 8410 includes a proximal end (not shown) that is coupled to the mechanical structure, and a distal end 8412 (see FIGS. 19 and 20) that is coupled to the beam 8810 via an anchor 8925 of the beam 8810. In some embodiments, the proximal end of the shaft 8410 is coupled to the mechanical structure in a manner that allows movement of the shaft 8410 along a center axis C-A of the shaft 8410 (shown in FIG. 19) relative to the mechanical structure. Allowing the shaft 8410 to “float” in the Z direction facilitates measurement of forces along the Z axis, as described herein. In some embodiments, the proximal end of the shaft 8410 can be movably coupled to the mechanical structure via a four bar linkage of the types shown and described in International Patent Appl. No. PCT/US2019/061883 (filed Nov. 15, 2019), entitled “Surgical Instrument with Sensor Aligned Cable Guide,” which is incorporated herein by reference in its entirety. The shaft 8410 also defines a lumen (not shown) and/or multiple passageways through which the cables and other components (e.g., electrical wires, ground wires, or the like) can be routed from the mechanical structure to the wrist assembly 8500. In this embodiment, a distal portion of the shaft 8410 is received at least partially within an interior region 8931 of the anchor 8925 and can be fixedly coupled to the anchor 8925 via an adhesive bond, a weld, or any other permanent coupling mechanism (i.e., a coupling mechanism that is not intended to be removed during normal use).

The outer shaft 8910 can be any suitable elongated shaft that can be disposed over the shaft 8410 and includes a proximal end (not shown) that can be coupled to the mechanical structure and a distal end 8912. The outer shaft 8910 defines a lumen between the proximal end and the distal end 8912. The shaft 8410 extends within the lumen of the outer shaft 8910 and the inner shaft 8410 can move relative to each other. For example, the shaft 8410 can rotate relative to the outer shaft 8910 and/or can translate longitudinally in a direction parallel to the center axis C-A of the shaft 8410. For example, in some embodiments, the proximal end of the outer shaft 8910 is fixedly coupled to the mechanical structure and the shaft 8410 is coupled to move relative to the mechanical structure. In other embodiments, the outer shaft 8910 or portions thereof can move relative to the mechanical structure (e.g., the outer shaft 8910 can be a telescoping shaft). Thus, in some embodiments, the outer shaft 8910 is operatively coupled to the mechanical structure and can be moved or translated longitudinally relative to the shaft 8410 in a direction parallel to the center axis C-A.

The mechanical structure produces movement of the cables (not shown) to produce the desired movement (pitch, yaw, or grip) at the wrist assembly 8500. Specifically, the mechanical structure includes components and controls to move some of the cables in a proximal direction (i.e., to pull in certain cables) while simultaneously allowing the distal movement (i.e., releasing or “paying out”) of other of the cables in equal lengths. In this manner, the mechanical structure can maintain the desired tension within the cables, and in some embodiments, can ensure that the lengths of the cables are conserved (i.e., moved in equal amounts) during the entire range of motion of the wrist assembly 8500. In other embodiments, however, conservation of the lengths of the cables is not required.

In some embodiments, the mechanical structure can include one or more mechanisms that produce translation (linear motion) of a portion of the cables. Such a mechanisms can include, for example, a gimbal, a lever, or any other suitable mechanism to directly pull (or release) an end portion of any of the cables. For example, in some embodiments, the mechanical structure can include any of the mechanical structures (referred to as backend assemblies or actuators) or components described in U.S. Patent Application Pub. No. US 20157/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disk Wrist Joint,” each of which is incorporated herein by reference in its entirety. In other embodiments, however, the mechanical structure can include a capstan or other motor-driven roller that rotates or “winds” a portion of any of the bands to produce the desired band movement. For example, in some embodiments, the mechanical structure can include any of the mechanical structures (referred to as backend assemblies or actuators) or components described in U.S. Pat. No. 9,204,923 B2 (filed Jul. 16, 2008), entitled “Medical Instrument Electronically Energized Using Drive Cables,” which is incorporated herein by reference in its entirety.

The wrist assembly 8500 includes a proximal first link 8510 and a distal second link 8610. The first link 8510 includes a distal portion that is coupled to a proximal portion of the second link 8610 at a joint such that the second link 8610 can rotate relative to the first link 8510 about the first axis of rotation A₁ (which functions as the pitch axis, the term pitch is arbitrary). The proximal first link 8510 includes a proximal portion that is coupled to the beam 8810 as described in more detail below.

A distal end of the distal second link 8610 is coupled to the end effector 8460 such that the end effector 8460 can rotate about the second axis of rotation A₂ (which functions as the yaw axis (the term yaw is arbitrary)). The end effector 8460 can include at least one tool member 8462 with a contact portion configured to engage or manipulate a target tissue during a surgical procedure. For example, in some embodiments, the contact portion can include an engagement surface that functions as a gripper, cutter, tissue manipulator, or the like. In other embodiments, the contact portion can be an energized tool member that is used for cauterization or electrosurgical procedures. The end effector 8460 is operatively coupled to the mechanical structure such that the tool member 8462 rotates relative to shaft 8410 about the first axis of rotation A₁. In this manner, the contact portion of the tool member 8462 can be actuated to engage or manipulate a target tissue during a surgical procedure. The tool member 8462 (or any of the tool members described herein) can be any suitable medical tool member. Moreover, although only one tool member 8462 is identified, as shown, the instrument 8400 can include two tool members that cooperatively perform gripping or shearing functions. In other embodiments, an end effector can include more than two tool members.

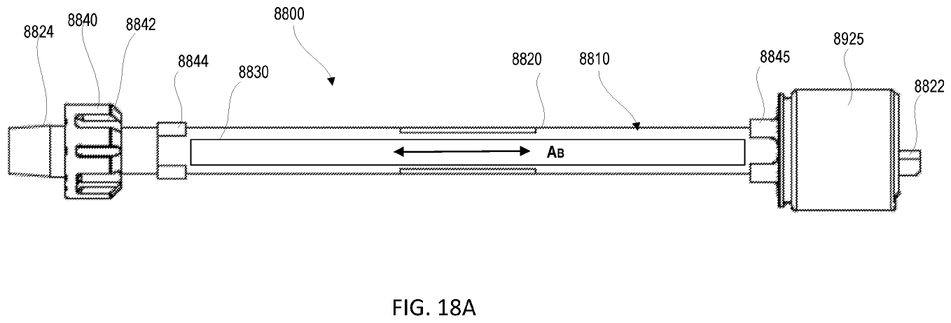

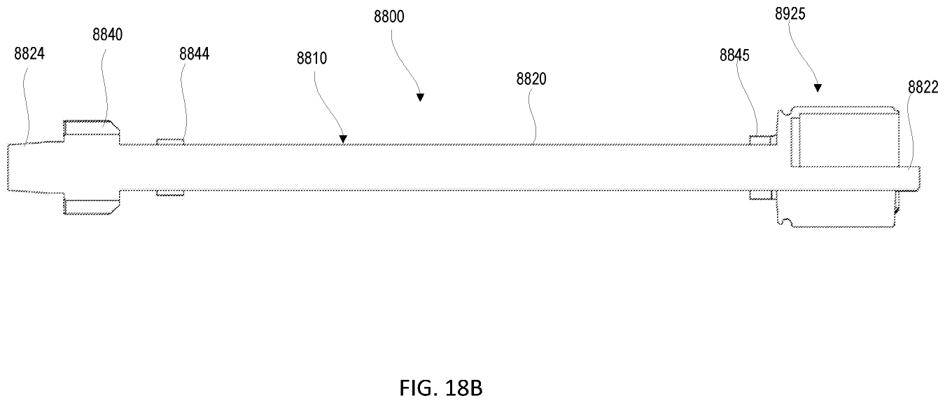

The beam 8810 includes a proximal end portion 8822, a middle portion 8820 (which functions as an active portion of the beam 8810) and a distal end portion 8824. The beam 8810 defines a center axis A_{B} defined along a length of the beam 8810 (see FIG. 18A). A strain sensor(s) 8830 (shown in FIG. 18A) is mounted on the middle portion 8820 of the beam 8810. The strain sensor 8830 is not shown in some of the figures for illustrative purposes only. In this embodiment, the strain sensor 8830 is a foil strain gauge. The strain sensor 8830 can be used to measure forces imparted on the surgical instrument during a surgical procedure as described in more detail below. In this embodiment, the middle portion 8820 defines four side surfaces disposed perpendicular to each other and the strain sensor 8830 is mounted on one of the side surfaces. In this embodiment, the cross-section of the middle portion 8820 is substantially square shaped. Thus, the cross-sectional shape of the middle portion 8820 is identical for every ninety degrees of rotation. In this manner, the output from the strain sensor 8830 will be consistent throughout the entire range of the roll of the shaft 8410 (i.e., rotation of the shaft 8410 about the center axis A_{B}).

In this embodiment, the beam 8810 includes the anchor 8925 at the proximal end portion 8822 of the beam 8810 and a collar 8840 near the distal end portion 8824 of the beam 8810. Thus, in this embodiment, the beam 8810 is formed integrally with the anchor 8925 and the collar 8840. The distal end portion 8824 is tapered and the proximal end portion 8822 extends through the anchor 8925. The middle portion 8820 of the beam 8810 includes tabs 8844 and 8845 that serve as guides for alignment between the middle portion of the beam 8810 and the strain sensor 8830. In some embodiments, either (or both) of the collar 8840 and the anchor 8925 can be manufactured separately and then later coupled to the middle portion 8820 of the beam 8810.

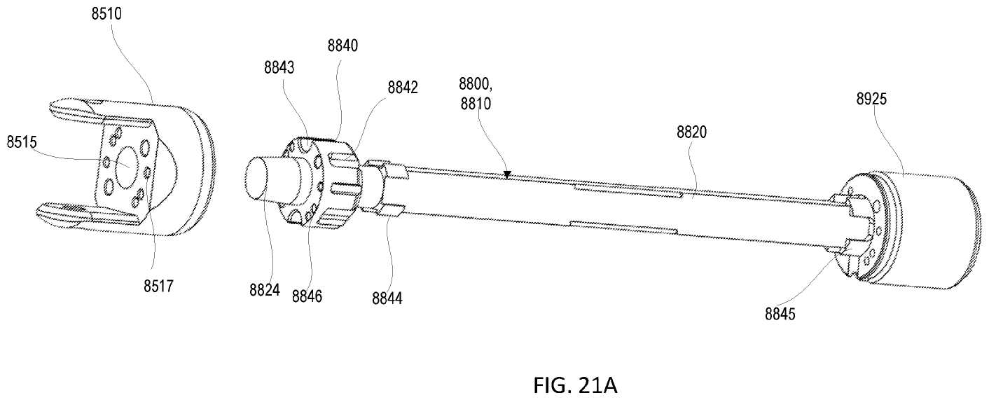

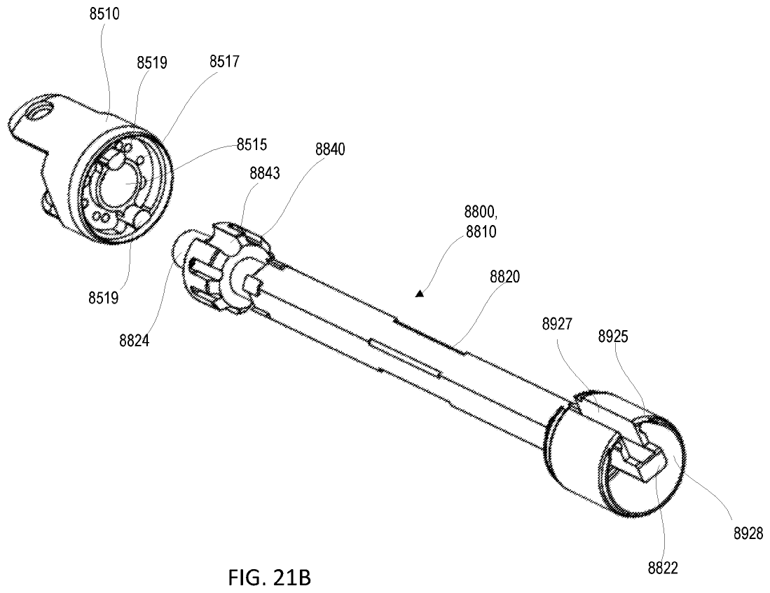

As described above, the beam 8810 is coupled to the distal end portion 8412 of the shaft 8410 via the anchor 8925. The beam 8810 is coupled to the proximal link 8510 of the wrist assembly 8500 via the distal end portion 8824 of the beam 8810. In this embodiment, the collar 8840 defines multiple slots 8842 in fluid communication with openings 8846 on a distal side of the collar 8840 similar to the slots 7513 for medical instrument 7400. The slots 8842 receive therethrough the cables (not shown) for actuating the wrist assembly 8500 and end effector 8460. For example, the cables can extend from the wrist assembly 8500, through openings 8517 of the proximal link 8510 (see FIG. 21A), through the openings 8846 and clots 8842.

The slots 8842 can also be used to introduce a fluid to effectuate cleaning of the instrument 8400. For example, when the outer shaft 8910 is moved to a distal position covering the beam 8810 and abutting the collar 8840, the distal end portion of the slots 8842 are exposed or accessible such that a cleaning fluid can be introduced through the distal end portion of the slots 8842. When the outer shaft 8910 is moved proximally, the slots 8842 and cables therein are also accessible for cleaning purposes. The collar 8840 also defines cutout portions 8843 and a center opening 8515. The cutout portions 8843 of the collar 8840 matingly couple with protrusions 8519 (see FIG. 21B) of the proximal link 8510, and the distal end portion 8824 of the beam 8810 is received within the center opening 8515 when the beam 8810 is coupled to the proximal link 8510. The distal end portion 8824 can be fixedly coupled to the proximal link 8510 within the center opening 8515 with, for example, one or more welds or adhesive or other suitable coupling method. The coupling of the cutout portions 8843 to the protrusions 8519 prevents rotation of the proximal link 8510 and the distal end portion 8824 of the beam 8810 relative to each other during assembly.

The anchor 8925 defines multiple openings 8929 that receive therethrough electrical wiring (for example, for powering a cauterizing tool of the end effector) and/or the cables extending from the collar 8840 for actuating the wrist assembly 8500 and effector 8460. For example, the larger openings 8929 can be used for the electrical wiring and the smaller openings 8929 can be used for the cables. The anchor 8925 also defines a slot 8927 through which the wiring for the sensor(s) 8830 can be routed. For example, the wiring can extend from the sensor(s) 8830 on the beam 8810 through the slot 8927, through the interior lumen of the shaft 8410 and to the mechanical structure at the proximal end of the medical instrument 8400.

In use, the end effector 8460 contacts anatomical tissue, which may result in X, Y, or Z direction forces (see FIG. 19) being imparted on the end effector 8460 and that may also result in moment forces about the various axes. As described above for instrument 2400, the strain sensor 8830 can be used to pick up signals induced by forces in the X, Y and Z directions and the signals can be used to measure strain in the beam 8810, which can in turn be used to determine forces imparted on the end effector 8460 in the X and Y axes directions. Thus, the strain sensor 8830 can be used to determine forces imparted on the end effector 8460 that are transverse (e.g., perpendicular) to the center axis A_{B} of the beam 8810 as such forces are transferred to the beam 8810 in the X and Y directions (see FIG. 19). Specifically, the transverse forces acting upon the end effector 8460 can cause a slight bending of the beam 8810, which can result in a tensile strain imparted to one side of the beam 8810 and a compression strain imparted to the opposite side of the beam 8810. The strain sensor 830 is coupled to the beam 8810 to measure such tensile and compression strains.

As described above, such X and Y forces can result in stress concentrations and strain overloads at the interface between the beam 8810 and its mating component, the proximal link 8510. Such stress concentrations and strain overloads can result in inaccurate readings at the force sensor 8830. As described herein, to increase the accuracy of the force measurements and reduce or eliminate such issues, a discontinuity is formed between the beam 8810 and the connection interface between the beam 8810 and its mating components. In this embodiment, a discontinuity is created by the connection of the beam 8810 to the link 8510, but because the anchor 8925 is integral with the beam 8810, there is no need to create a discontinuity at the proximal end portion 8822 of the beam 8810. More specifically, as described above, the distal end portion 8824 has a different cross-sectional area than a cross-sectional area of the middle portion 8820 of the beam 8810 on which the force sensor 8830 is mounted. This creates a discontinuity between the distal end portion 8824 and the middle portion 8820 and provides for stress concentrations or strain overloads to be transferred to the distal interface where the distal end portion 8824 is coupled to the link 8510.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, cables, or other components described herein can be constructed from multiple pieces that are later joined together. For example, in some embodiments, a link can be constructed by joining together separately constructed components. In other embodiments however, any of the links, tool members, beams, shafts, cables, or components described herein can be monolithically constructed.

Although the instruments are generally shown as having an axis of rotation of the tool members (e.g., axis A₂) that is normal to an axis of rotation of the wrist member (e.g., axis A₁), in other embodiments any of the instruments described herein can include a tool member axis of rotation that is offset from the axis of rotation of the wrist assembly by any suitable angle.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A medical device comprising:
a shaft comprising a proximal end portion and a distal end portion;
a beam comprising a proximal end portion, a distal end portion, and a middle portion between the proximal end portion of the beam and the distal end portion of the beam; and
one or more strain sensors on the middle portion of the beam;
wherein the proximal end portion of the beam is matingly coupled to the distal end portion of the shaft to form an interface; and
the beam comprises a discontinuity between the interface and the middle portion of the beam.

2. The medical device of claim 1, wherein:
the interface is a first interface, and the discontinuity is a first discontinuity;
the medical device comprises a link;
the distal end portion of the beam is matingly coupled to the link to form a second interface; and
the beam comprises a second discontinuity between the second interface and the middle portion of the beam.

3. The medical device of claim 2, wherein:
the middle portion of the beam has a first cross-sectional area at the first discontinuity;
the proximal end portion of the beam has a second cross-sectional area at the first discontinuity;
the distal end portion of the beam has a third cross-sectional area at the second discontinuity; and
the first cross-sectional area is different than the second cross-sectional area and the third cross-sectional area.

4. The medical device of claim 2, wherein:
a center axis of the shaft is defined between the proximal end portion of the shaft and the distal end portion of the shaft;
the proximal end portion of the beam comprises a tapered guide for alignment of the beam to the distal end portion of the shaft along the center axis of the shaft; and
the distal end portion of the beam comprises a tapered guide for alignment of the beam to the link along the center axis of the shaft.

5. The medical device of claim 2, wherein:
the first discontinuity comprises a first recessed region between the middle portion of the beam and the proximal portion of the beam; and
the second discontinuity comprises a second recessed region between the middle portion of the beam and the distal end portion of the beam.

6. The medical device of claim 1, wherein:
the distal end portion of the shaft comprises an anchor;
the anchor comprises a coupling portion; and
the proximal end portion of the beam is matingly coupled to the coupling portion of the anchor to couple the distal end portion of the beam to the distal end portion of the shaft.

7. The medical device of claim 6, wherein:
the anchor comprises an opening;
the opening comprises a shape; and
the proximal end portion of the beam comprises a shape to be received in only one orientation within the shape of the opening of the anchor.

8. The medical device of claim 1, wherein:
the middle portion of the beam comprises a proximal end face and a distal end face;
the anchor comprises a distal end face;
the link comprises a proximal end face;
the proximal end face of the middle portion of the beam is spaced apart from the distal end face of the anchor when the beam is coupled to the anchor; and
the distal end face of the middle portion of the beam is spaced apart from the proximal end face of the link when the beam is coupled to the link.

9. A medical device comprising:
a shaft comprising a proximal end portion, a distal end portion, and a center axis extending between the proximal end portion and the distal end portion;
a beam comprising a proximal end portion, a distal end portion, and a middle portion between the proximal end portion and the distal end portion,
the proximal end portion of the beam having a first cross-sectional area,
the distal end portion of the beam having a second cross-sectional area different from the first cross-sectional area, and
the middle portion of the beam having a third cross-sectional area different from the first cross-sectional area;
one or more strain sensors on the middle portion of the beam;
an anchor coupled to the distal end portion of the shaft,
the anchor comprising a coupling portion, and
the proximal end portion of the beam being matingly coupled to the coupling portion of the anchor; and
a link comprising a coupling portion, the distal end portion of the beam being matingly coupled to the coupling portion of the link.

10. The medical device of claim 9, wherein:
the coupling portion of the anchor comprises an opening configured to receive the proximal end portion of the beam; and
the coupling portion of the link comprises an opening configured to receive the distal end portion of the beam.

11. The medical device of claim 9, wherein:
the proximal end portion of the beam is a tapered proximal end portion, and the distal end portion of the beam is a tapered distal end portion;
the tapered proximal end portion is a guide for alignment of the beam to the anchor along the center axis of the shaft; and
the tapered distal end portion is a guide for alignment of the beam to the link along the center axis of the shaft.

12. The medical device of claim 9, wherein:
the anchor comprises an opening;
the link comprises an opening;
the proximal end portion of the beam comprises a shape to be received in only one orientation within the opening of the anchor; and
the distal end portion of the beam comprises a shape to be received in only one orientation within the opening of the link.

13. The medical device of claim 9, wherein:
the anchor comprises a D-shaped opening;
the link comprises a D-shaped opening;
the proximal end portion of the beam comprises a D-shape configured to be received within the D-shaped opening of the anchor; and
the distal end portion of the beam comprises a D-shape configured to be received within the D-shaped opening of the link.

14. The medical device of claim 9, wherein:
the proximal end portion of the beam is welded to the anchor; and
the distal end portion of the beam is welded to the link.

15. The medical device of claim 9, wherein:
the medical device comprises an outer shaft;
the outer shaft comprises a lumen;
the shaft extends within the lumen of the outer shaft; and
the shaft is movable relative to the outer shaft.

16. The medical device of claim 15, wherein:
the outer shaft translates longitudinally along the center axis relative to the shaft and the beam.

17. The medical device of claim 15, wherein:
at least one of the shaft and the outer shaft rotates relative to the other of the shaft and the outer shaft.

18. The medical device of claim 9, wherein:
the one or more strain sensors include a first strain sensor and a strain second sensor;
the first strain sensor is on the middle portion of the beam at a first location on the middle portion of the beam; and
the second strain sensor is on the middle portion of the beam at a second location on the middle portion of the beam spaced apart from the first strain sensor in a direction along a length of the beam.

19. The medical device of claim 18, wherein:
the middle portion of the beam comprises a first outer face, a second outer face, a third outer face, and a fourth outer face;
the third cross-sectional area of the middle portion of the beam has a length along the first and third outer faces, and a width along the second and fourth outer faces;
the first outer face and the second outer face are oriented perpendicular to the third outer face and the fourth outer face; and
the first strain sensor and the second strain sensor are each on the first outer face.

20-26. (canceled)

27. The medical device of claim 9, wherein:
the first cross-sectional area of the proximal end portion of the beam and the second cross-sectional area of the distal end portion of the beam are each smaller than the third cross-sectional area of the middle portion of the beam.
